(19) United States
(12) Patent Application Publication
(10) Pub. No.: US 2006/0286142 A1
(43) Pub. Date: Dec. 21, 2006

(54) Gold surfaces coated with a thermostable chemically resistant polypeptide layer and applications thereof

(75) Inventors: Richard G. Woodbury, Seattle, WA (US); Theo deVos, Seattle, WA (US); Meher Irani, Edmonds, WA (US); James Clendenning, Seattle, WA (US)

Correspondence Address: DLA PIPER US LLP, 4365 EXECUTIVE DRIVE, SUITE 1100, SAN DIEGO, CA, 92121-2133, US

(73) Assignee: BIOHESION, INC., Seattle, WA (US)

(21) Appl. No.: 11/446,509

(22) Filed: Jun. 2, 2006

Related U.S. Application Data
(60) Provisional application No. 60/686,554, filed on Jun. 3, 2005.

(51) Int. Cl. A61F 2/02; A61K 9/28; C12M 1/34; B05D 3/02

(52) U.S. Cl. 424/423; 435/287.1; 427/2.11; 427/2.14; 977/900

(57) ABSTRACT

The present invention provides a method for producing biomolecular coatings on devices having gold surfaces. The method describes the production of recombinant fusion proteins consisting of one or more polypeptide domains of interest and a high affinity gold binding peptide consisting of 1 to 7 repeats of a gold binding protein (GBP) sequence. By this method, many biologically active polypeptides lacking intrinsic gold-binding properties can be firmly attached to gold surfaces. By exploiting such gold binding properties, devices are disclosed which comprise such coatings that are useful as prosthetic devices, implants, and tissue interfacing materials. Further, such devices comprising these coatings protect surfaces from fouling and impart various properties to the coated devices.

### RELATED APPLICATION

This application claims benefit to U.S. Provisional Application No. 60/686,554, filed on Jun. 3, 2005, the entire contents of which is incorporated herein by reference.

### STATEMENT OF GOVERNMENT SUPPORT

This invention was made in part with government support under Grant No. 1 R43 EB000931-01A1 awarded by The National Institutes of Health. The government has certain rights in this invention.

### BACKGROUND OF THE INVENTION

1. Field of the Invention

The present invention relates generally to the production of biomolecular coatings and more specifically to methods for modifying gold containing surfaces of devices, including such devices, where the coatings comprises gold binding protein domains.

2. Background Information

Robust attachment of proteins and other macromolecules, e.g., recognition or affinity-binding molecules or enzymes, to a surface such as gold is an important step in implementing a variety of technologies including the development of biomaterials and biosensors. Gold is an excellent material for introducing surface functionality via the attachment of proteins or other macromolecules because of the metal's chemical inertness, electrical conductivity, surface uniformity and stability, biologic compatibility/low toxicity and other properties. Gold's chemical inertness, however, limits the ability to prepare functional surfaces to just a few proteins or other macromolecules that produce stable biomaterial/biomolecular coatings when adsorbed directly onto a clean gold surface. Moreover, it seems that only large molecules such as proteins, proteoglycans, or structures such as membrane-bound lipids typically bind well to gold.

By definition, a biomaterial is a nonviable material used in a medical device, intended to interact with or be in contact with biological systems. By way of relevant example, gold fillings are a classic biomaterial. Although they are primarily recognized for medical/dental applications, biomaterial uses range from cell culture, to devices, to assay blood proteins in the clinical laboratory, heart-lung machines that support blood flow during surgery, kidney dialysis machines, to implantable ID tags for pets. The common feature amongst the different applications is the interaction between biological factors and processes and the synthetic or altered natural materials (biomaterial).

Medical implants include dental, hip, knee, and heart valve replacement, and inserted devices such as coronary stents, stimulatory electrodes, pumps, and urinary catheters. Other devices such as kidney dialysis and heart-lung machines operate in contact with biological fluids and secretions. Other biomaterials are being developed for drug-delivery and contrast agents in bioimaging. In many instances, the effectiveness of the device can be enhanced by attaching certain bioactive molecules to the surface of the device. For example, orthopedic implants are significantly more effective when coated with human bone sialoprotein (BSP) and/or osteopontin (OPN), two proteins that facilitate osteoblast adherence to implants which leads to enhanced osseointegration. These proteins contain an Arg-Gly-Asp (RGD) sequence common to many connective tissue proteins that interacts with integrin receptors on cell surfaces to allow attachment to biomaterials. BSP also contains a putative heparin binding motif that could relate to protein function. A consensus sequence motif—Phe-His-Arg-Arg-Ile-Lys-Ala—for heparin binding has been reported. There are also regions of OPN and BSP proteins that have aspartic acid- or glutamic acid-rich sequences that may sequester or concentrate calcium to foster mineralization. Increasing phosphorylation of OPN and BSP, also, appears to correlate with the activities of these proteins in mineralization. In such instances where enhancement is exploited through these proteins, longer persistence of the bioactive molecule on the surface can be important to successful operation of the device.

Other proteins including bone morphogenetic protein (BMP) and related TGF-beta1, osteonectin (ON), osteocalcin (OC), fibronectin (FN), type I collagen, and fibroblast growth factors (FGF-1 and FGF-2) are also important in bone development. Generally, if such proteins are physically adsorbed to implant material, they quickly wash away when placed in tissue. Consequently, there is much effort to develop methods that increase the time of persistence of bioactive molecules at implantation sites.

Equally important to the successful use of biomaterials, is the minimization of the foreign body reaction mounted by the body toward the device. This process is stimulated initially by the adsorption of plasma or blood proteins, e.g., fibrinogen, to the surface of the biomaterial, and later by cellular (platelets and fibroblasts) and tissue defenses (neutrophils and macrophage) that can include inflammation. Frequently, the biomaterial is encapsulated by collagen and fibroblasts in an attempt to isolate the material from healthy tissue. Encapsulation generally means the device will fail. Also, implants frequently foster bacterial infections. A major goal in biomaterial development, then, is the elimination of surface fouling in the presence of body fluids and tissues and acceptance of the biomaterial as a natural element.

Paradoxically, the same healing factors attached to implants can have a negative effect on osseointegration and healing, if present in too high a concentration or when the factors persist too long on an implant. Typically, factors that bind and stimulate osteoblasts that facilitate bone mineralization can also activate osteoclasts that lead to bone resorption. While the presence of healing factors can facilitate successful implantation, the relative surface roughness and irregular shape of implants, also, appears to increase cell adhesion and osseointegration. Therefore, the surface of an “ideal” biomaterial will promote rapid healing and anchoring in bone or other tissue while simultaneously eliminating undesirable surface fouling leading to foreign body reactions, infection, and bone resorption.

Biodetection refers to the quantitative measurement of biological substance in a sample. For example, most clinical diagnostic tests are based on Biodetection. Enzyme-Linked ImmunoSorbent Assay (ELISA) testing is the predominate biodetection system used today. When adapted for specific testing it is a powerful and sensitive approach for the diagnostic detection of many biological targets. In testing for infectious diseases and other clinical indications it has been the gold standard for two decades in healthcare. The ELISA approach, also, is widely used in research and drug discovery. The tests are based on specific antibodies that attach to target molecules that are present in samples. Attached to the antibodies are certain enzymes that produce a colored product that can be quantitatively measured. The amount of color produced depends on how much antibody is attached to target molecules and, therefore, color development is proportional to the amount of target in samples.

While ELISA testing is the current workhorse in biodetection, presently there is much activity and investment to develop alternative diagnostic approaches. The drivers include faster test results, more user-friendly operation, lower cost, and a big demand for point-of-care testing (PoCT). ELISA testing requires highly-skilled operators, costly reagents, typically 4 to 6 hours for results, and large, expensive supporting instruments/computers for analysis of tests. Therefore, ELISA testing occurs almost exclusively in centralized clinical and research laboratories and, thus, does not address the urgent need for PoCT. Consequently, there is much effort and investment in R&D to develop rapid diagnostic tests for PoCT. Of particular interest in this area, is the development of real-time testing platforms that can take the place of ELISA testing and other clinical tests conducted in centralized reference laboratories.

Diagnostic testing for various analytes and monitoring of certain processes are important in industry, food safety, bioremediation, environmental assessment, and detection of bioterrorist agents. A major goal in this area is to achieve real-time or on-line analysis that can eliminate the requirement of inefficient off-site analysis in centralized reference laboratories. However, the prevailing conditions under which testing or monitoring occurs can be extremely variable and harsh making it difficult to obtain reliable results.

These non-medical applications, nonetheless, are often enabled by bioactive molecules that permit biodetection much in the same way as discussed above and the requirements for efficient performance are similar. Commercial applications are possible when conditions allow high bioactivity. However, many applications are not possible because of extreme or variable conditions that destroy bioactivity directly or indirectly because the bioactivity dissociates from the detection surface.

In recent years, it has become apparent that certain microorganisms, e.g., bacteria, can survive at extremely high temperatures or under other extreme conditions, such as high/low salinity or pH. For example, thermophilic organisms thrive in high temperature environments. Many of the enzymes and other bioactive molecules found in mesophilic organisms have similar counterparts in thermophiles that have identical functions and similar 3-D shapes. Amino acid sequences of the enzyme analogues, however, are significantly different in regions that confer stability. Other bioactive molecule analogues in mesophiles and thermophiles, e.g., lipids and carbohydrates, are also chemically distinct.

Other forms of extremophilic organisms can live in highly acidic environments, or environments that contain high concentrations of sulfur compounds, or in high salt environments. Many extracellular or secreted enzymes and other biomolecules from these organisms can function in these extreme environments. Bioactive molecules from extremophiles can have industrial, environmental monitoring, bioremediation applications not possible using mesophilic analogue molecules.

There is much potential for using thermophilic enzymes in industrial or bioremediation processes at elevated temperature. Compared to catalysis at ambient temperature and up to 37° C. the benefits of catalysis at high temperature include: accelerated catalysis; increased solubility of many compounds; higher diffusion rates of reactants; decreased solution viscosity to benefit flow processes; and removal of volatile compounds.

Temperatures generally must exceed 60°-70° C. for optimum thermophilic enzyme activity. Unless covalently attached to detection surfaces, the enzymes can dissociate from the surface at these temperatures. Foundation layers on the surface used to covalently attach bioactivity can be disrupted at high temperatures. Similarly, other extreme conditions can negatively affect the stability of bioactive layers on detection surfaces.

### SUMMARY OF THE INVENTION

The present invention discloses a method to achieve robust, efficient immobilization of biomolecules to gold containing surfaces of devices regardless of the intrinsic capacity of the biomolecule to bind gold directly. The invention can be applied to fabricate coatings for biomaterials designed for tissue interfacing, clinical, environmental testing, and industrial applications. The present invention can greatly expand the number of potential applications that are based on biomaterial deposition on gold surfaces. The invention discloses recombinant fusion proteins capable of immobilizing biomolecules on a desired gold containing surface, including the generation of monolayers on such surfaces. This is accomplished by fusion proteins comprising a gold-binding peptide (GBP) domain as the agent for immobilization. In certain embodiments, appropriate conditions allow selective binding of GBP to the desired surface while minimizing surface interaction with biomolecule comprising the fusion protein. Fusion proteins, e.g., comprising thermophilic/extremophilic enzymes, can be tethered from the gold surface into solution with retention of up to 100% of activity when exposed to high temperatures.

In one embodiment, a method of forming a biomolecular coating on a surface of a medical device is disclosed including providing a medical device, where the device has one or more gold surfaces and applying a biomaterial to the device, where the biomaterial is adsorbed on or is formed on a surface thereof, and where the biomaterial includes a fusion protein having at least one gold binding protein (GBP) domain and at least one proteinaceous biomolecule domain, where applying the biomaterial immobilizes the biomolecule on the surface, thereby forming a biomolecular coating on the medical device.

In one aspect, the biomolecule imparts biocompatibility characteristics to the surface of the device. In another aspect, the biomolecule promotes tissue healing and repair. In another aspect, the coating imparts resistance to fouling of the surface of the device.

In a related aspect, at least one biomolecule is selected from the group consisting of an anti-thrombotic protein, an anti-inflammatory protein, an antibody, an antigen, an immunoglobulin, an enzyme, a hormone, a neurotransmitter, a cytokine, a protein, a globular protein, a cell attachment protein, a peptide, a cell attachment peptide, a toxin, an antimicrobial protein, and a growth factor. In a related aspect, the biomolecule is bone sialoprotein (BSP) or osteopontin (OPN).

In one aspect, such devices include, but are not limited to, a blood-contacting medical device, a tissue-contacting medical device, a bodily fluid-contacting medical device, an implantable medical device, an extracorporeal medical device, a dental device, a dental implant, a blood oxygenator, a blood pump, tubing for carrying blood, an endoprosthesis medical device, a vascular graft, a stent, a pacemaker lead, a heart valve, a temporary intravascular medical device, a catheter, and a guide wire.

In another embodiment, a tissue-interface device is disclosed, including at least one gold surface, which surface is routinely exposed to a tissue of a subject, and a biomaterial adsorbed on or formed on the surface to be exposed, where the biomaterial comprises a fusion protein having at least one gold binding protein (GBP) domain and at least one proteinaceous biomolecule domain, and where the adsorbed biomaterial immobilizes the biomolecule on the surface of the device.

In one aspect, the biomolecule imparts biocompatibility characteristics to the surface of the device. In another aspect, the biomolecule promotes tissue healing and repair. In another aspect, the coating imparts resistance to fouling of the surface of the device.

In one embodiment, a method of sterilizing a gold containing device including applying a biomaterial coating on the device, where the biomaterial is adsorbed on or is formed on a surface of the device, and where the biomaterial comprises a fusion protein having at least one gold binding protein (GBP) domain and sterilizing the coated device by a process including: exposing the device to organic solutions selected from the group consisting of Gu-HCl, Triton X-100, methanol, ethanol, isopropanol, urea, acetic acid, and glycine-HCl, exposing the device to strong acids or bases, exposing the device to a temperature of about 100° C., exposing the device to solutions of high ionic strength, or a combination of the processes, where the sterilizing does not significantly impact the adsorption of the GBP domain to the surface of the device.

In one aspect, the GBP imparts biocompatibility characteristics to the surface of the device. In another aspect, the fusion protein comprises a thermophilic or extremophilic enzyme. In a related aspect, the enzyme includes, but is not limited to, RNases, polymerases, restriction endonuc leases, reductases, amino transferases, dismutases, synthases, amino peptidases, kinases, ligases, proteases, carboxypeptidases, phosphatases, binding proteins, amylases, pullulanases, amylopullulanases, glucoamylases, CGTases, glucanases, cellobiohydrolases, endoxylanases, mannanases, xylosidases, glucosidases, hydantoinases, esterases, aldolases, cytochrome P450, dehydrogenases, methylesterases, lyases, galactosidases, fructosidases, endoglucanases, phytases, keratinases, chitinases, and isomerases.

In one embodiment, a method of adsorbing a thermophilic or extremophilic enzyme to a gold containing surface is disclosed including providing one or more gold surfaces, and adsorbing a biomaterial on the one or more surfaces, where the biomaterial is adsorbed on or is formed on one or more surfaces, and where the biomaterial comprises a fusion protein having at least one gold binding protein (GBP) domain and at least one domain comprising a thermophilic or extremophilic enzyme, where adsorbing the biomaterial immobilizes the thermophilic or extremophilic enzyme on the one or more surfaces.

In one aspect, the surface is regularly exposed to temperature ranges from about 40° C. to about 100° C. In a related aspect, the surface is selected from the group consisting of a bead, a microchip, an array, and a biosensor.

In one aspect, thermophilic enzymes include, but are not limited to, RNases, polymerases, restriction endonucleases, reductases, amino transferases, dismutases, synthases, amino peptidases, kinases, ligases, proteases, carboxypeptidases, phosphatases, and binding proteins.

In another aspect, extremophilic enzymes include, but are not limited to, amylases, pullulanases, amylopullulanases, glucoamylases, CGTase, glucanases, cellobiohydrolases, endoxylanases, mannanases, xylosidases, glucosidases, hydantoinases, esterases, aldolases, cytochrome P450, dehydrogenases, methylesterases, lyases, galactosidases, fructosidases, endoglucanases, phytases, keratinases, chitinases, and isomerases.

In another embodiment, a gold containing device is disclosed including fusion protein adsorbed to one or more gold surfaces comprising the device, where the fusion protein comprises at least one gold binding protein (GBP) domain and at least one domain comprising a thermophilic or extremophilic enzyme, and wherein the GBP domain immobilizes the thermophilic or extremophilic enzyme on the surface of the device.

In one embodiment, a method of providing a gold surface monolayer is disclosed including applying a binding partner on a planar surface, applying a fusion protein to the planar surface, where the fusion protein comprises a gold binding protein (GBP) domain and a protein domain, where the protein domain is a cognate binding partner to the applied binding partner, and exposing the bound planar surface to one or more modalities comprising one or more gold surfaces, where the modalities are selected from the group consisting of gold comprising beads, colloidal gold, gold powder, and gold comprising nanoparticles, where the interaction between the binding partner on the planar surface and cognate binding partner of the fusion protein drives the assembly of the modalities, thereby forming a gold containing monolayer on the planar surface.

In one aspect, the protein domain includes, but is not limited to, protein A, protein G, streptavidin, core streptavidin, neutravidin, avidin, avidin related protein 4/5, strep-tag, strep-tag II, an antibody, an antibody fragment, a single chain antibody, a receptor, and a peptide ligand.

In another aspect, the binding partner on the planar surface includes, but is not limited to, protein A, protein G, streptavidin, core streptavidin, neutravidin, avidin, avidin related protein 4/5, strep-tag, strep-tag II, an antibody, an antibody fragment, a single chain antibody, biotin, receptor ligands, small molecules, nucleic acids, carbohydrates, lipids, inorganic compounds, organic compounds, vitamins, metals, and peptide ligands.

A further understanding of the nature and advantages of the invention will become apparent from the detailed description, other specific examples of the invention, and other information provided below.

### BRIEF DESCRIPTION OF THE DRAWINGS

FIG. 1 shows a graph of results of 1.5 hour stability evaluation of GBP/gold complexes.

FIG. 2 shows a graph of results of 72 hour stability evaluation of GBP/gold complexes.

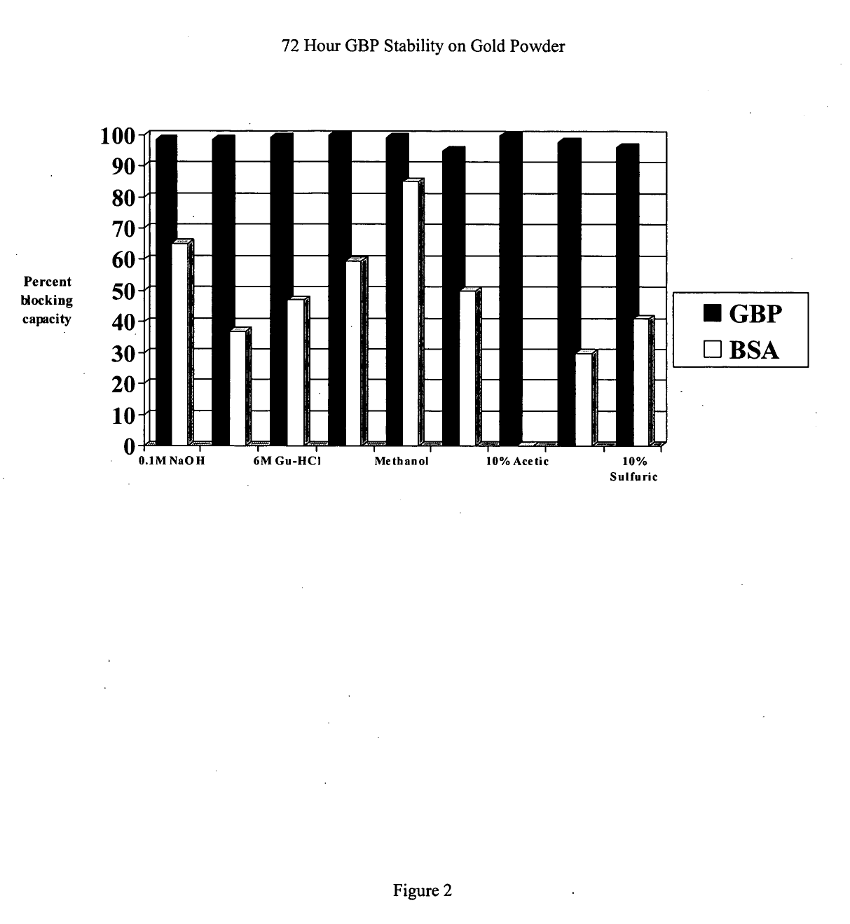

FIG. 3 illustrates data from SPR sensor experiments regarding GBP stability on gold.

FIG. 4 shows SPR data regarding non-fouling property of GBP/gold surface following incubation with human fibrinogen and human serum albumin, human whole plasma, or human platelet-enriched whole plasma.

FIG. 5 demonstrates the bioactivity of GBP-Streptavidin following incubation with human proteins and plasmas.

FIG. 6 illustrates how to derivatize gold biomaterials with OPN, BSP or other to biomolecules which impart bioactivity to surfaces.

FIG. 7 shows a table of Arg-Gly-Asp flanking sequences from various connective tissue proteins.

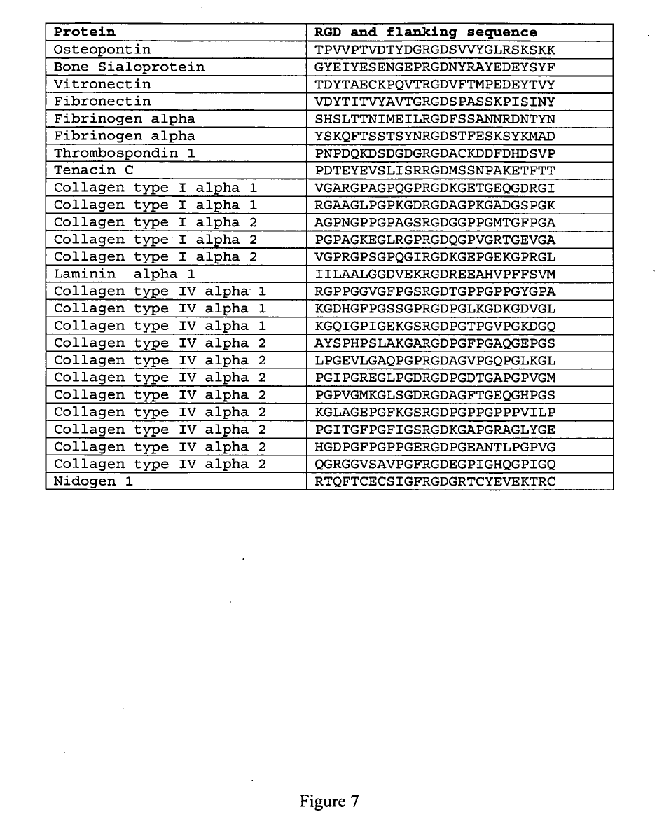

FIG. 8 shows a plasmid map depicting the expression vector for insertion of DNA encoding GBP fusion proteins.

### DETAILED DESCRIPTION OF THE INVENTION

Before the present compositions, methods, and devices are described, it is to be understood that this invention is not limited to particular compositions, methods, and devices described, as such compositions, methods, and device components may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference in their entirety.

The term “biomolecular coating,” including grammatical variations thereof, as used herein means a covering containing a naturally or non-naturally occurring chemical compound that modulates living cells or properties cellular components, which covering is applied to an object that modifies the properties of the surface of the object to which it is applied. For example, the coating may comprise a chemical which imparts biocompatibility properties to the surface of the object, or may impart tissue modulating properties to the surface of the object, or may protect the surface of the object from fouling caused by interfacing the surface with biological tissues.

The term “impart,” including grammatical variations thereof, as used herein means to give or convey.

The term “promote,” including grammatical variations thereof, as used herein means to help bring about.

The term “resistance,” including grammatical variations thereof, as used herein means to retard or oppose a particular effect (e.g., oppose attachment of plasma factors which foul tissue interfacing devices).

The term “tissue interface device,” including grammatical variations thereof, as used herein means a piece of equipment or a mechanism which comprises a surface that forms a common boundary between the equipment or mechanism and an aggregate of cells of a particular kind. For example, a needle on a syringe would be a tissue interface device. In a related aspect, the term “surface is routinely exposed to a tissue of a subject,” includes upper boundaries of an object which are part of equipment or mechanisms that are engaged with tissues as a regular course of their performance. In a further related aspect, “surface regularly exposed to temperature ranges,” is a similar surface that is exposed to such a temperature environment as a routine course of its performance.

The term “gold surface,” including grammatical variations thereof, as used herein means the exterior or upper boundary of an object or body characterized by resistance to deformation and to changes of volume that contain, comprise, or are coated with the element gold.

The term “proteinaceous” including grammatical variations thereof, as used herein means an amino acid sequence joined by peptide bonds which may be a full length protein or less than a full length protein or gene product, where the amino acid sequence making up the full length protein, less than full length protein, or gene product has a specific biochemical function (e.g., an enzyme or binding domain). In a related aspect, within a protein, a structural domain (“domain”) is an element of overall structure that is self-stabilizing and often folds independently of the rest of the protein chain. Most domains can be classified into “folds”. Many domains are not unique to the proteins produced by one gene or one gene family but instead appear in a variety of proteins, for example, the “calcium-binding” domain of calmodulin. Because they are self-stabilizing, domains can be “swapped” by recombinant techniques well known in the art between one protein and another to make chimeric proteins. A domain as used herein may be composed of none, one, or many structural motifs.

The term “sterilize,” including grammatical variations thereof, as used herein means to make substantially free of viable microbes. In a related aspect, “does not significantly impact the adsorption of the GBP domain to the surface of the device” includes resistance by gold bound GBP to release from the exterior surface to which it is attached.

The term “immobilized moiety,” including grammatical variations thereof, as used herein means a membrane bound compartment, chemical, mixture of chemicals, or mixture of molecules that are limited in their freedom of movement when such a compartment, chemical or mixture of chemicals are adsorbed on a solid phase. In a related aspect, an immobilized moiety includes, but is not limited to, peptide, a polypeptide, an organic molecule, an inorganic molecule, a nucleic acid, a lipid, a carbohydrate, a prokaryotic cell, a eukaryotic cell, a virus, or a combination thereof.

The term “substrate,” when referring to catalytic activity, means a substance acted upon by the active site of an enzyme.

The term “low complexity” as used herein means a few in number of different sequences.

The present invention discloses a method to achieve robust, efficient immobilization of biomolecules to gold containing surfaces regardless of the intrinsic capacity of the biomolecule to bind gold directly.

Further, the invention described herein produces recombinant fusion proteins comprising a unique GBP consisting of one or more repeats of the 14 amino acid sequence, Met-His-Gly-Lys-Thr-Gln-Ala-Thr-Ser-Gly-Thr-Ile-Gln-Ser (SEQ ID NO:1), and any desired polypeptide specifying activity, binding such fusion protein to a gold surface thereby introducing functionality to the surface.

A variety of materials, e.g., titanium alloys, platinum, stainless steel, plastics, polymers, ceramics, silicon, and others, have been used for medical implants and other biomaterials. Silane chemistry is quite effective for attaching bioactive molecules to those materials that contain oxides, e.g., titanium alloys.

In many ways gold is an ideal material for biomaterials and detection surfaces in biodetection. Pure gold is biologically inert in the body and appears non-cytotoxic (Shukla, et al., Langmuir 21:10644-10654, 2005; Hainfield, et al., Br. J. Radiol. 79:248-253, 2006; Rosi, et al., Science 312:1027-1030, 2006). Certain substances, e.g., proteins, may adhere to gold by hydrophobic or hydrophilic interaction, but generally the attraction is weak compared to association through covalent bonds. The chemical, electrical, and physical properties of gold make it the material of first choice for biomaterial and biodetection, if appropriate bioactive molecules can be securely attached to the metal's surface. Derivatizing gold surfaces can be a serious challenge, however, considering that pure gold is chemically resistant, especially to oxidative reactions often used on metals. Few molecules naturally bind strongly to pure gold that lacks a surface charge. Some forms of gold, e.g., colloidal gold (CG), are prepared from gold salts containing chloride or citrate anions resulting in particles with a negative surface charge. The lack of reactivity of uncharged gold makes it particularly useful in constructing biomaterials and detection surfaces that resist fouling. The same lack of reactivity of gold that provides resistance to surface fouling, however, significantly limits the use of gold in many applications because of the difficulty in securely attaching bioactive molecules to gold. Gold alloys, e.g., with silver, can increase chemical reactivity, but with a corresponding reduction in the non-fouling property of pure gold.

The GBP fusion proteins of the present invention show stability of complexes between GBP and gold under extreme chemical and physical conditions including high temperature, harsh chemicals, corrosive agents and solvents, or extreme pH. In one embodiment, devices comprising GBP on gold are resistant to proteolysis by the enzyme trypsin and appears to resist surface fouling when exposed to high concentration of various proteins, including human fibrinogen and serum albumin, which demonstrates that GBP binds to gold to form a monolayer that, for all practical purposes, is permanently attached to the surface.

The present invention discloses that GBP provides an efficient barrier that protects gold surfaces from the major blood proteins that typically bind to unprotected surface material used in medical implants.

The extracellular matrix (ECM) of tissues provides essential functions to cells leading to healthy cells, tissues and organs. The components of ECM are secreted by resident tissue cells and in turn the ECM sustains and protects the cells. ECM directly supports morphogenesis and wound healing of tissues by facilitating cell migration, attachment, spreading, stimulation, activity growth, and proliferation—in addition to providing a scaffold to accommodate and protect connective tissue cells (Albert, et al., In “Molecular Biology of the Cell” 4^{th} ed, pp 1090-1117, Garland Science, NY, N.Y., 2002).

In addition to myriad extracellular connective tissue proteins, ECM contains hyaluronic acid (HA) also known as hyaluronan (with MW range=150,000 to 6×106 Daltons) and large proteoglycan molecules that have long rigid chains of repeating disaccharides containing an amino sugar (N-acetylglucosamine or N-acetylgalactosamine) and D-glucuronic acid that together form glycosaminoglycans (GAG). The highly anionic HA due to carboxyl groups attracts large numbers of sodium ions in bodily fluids leading to a flow of water into the molecule and subsequent swelling of HA to form a hydrogel providing ECM with a permeable scaffold that has high turgor to resist compression. During tissue morphogenesis and repair, HA is one of the first molecules produced by proliferating, migrating cells. The permeable HA scaffold facilitates cell migration and subsequent secretion of other components of the ECM via specific pathways (Toole, J. Clin. Invest. 106:335-336, 2000).

HA does not contain a polypeptide core as do proteoglycans, however, the simple repeating GAG unit binds to specific cell-surface receptors, e.g., CD44, RHAMM, and LYVE-1, connective tissue protein sequence motifs, and recognition sites on proteoglycans (Bajorath, Proteins 39:103-111, 2000; Greiner, et. al., Exp. Hematol. 30:1029-1035; Jackson, Trends Cardiovasc. Med 13:1-7, 2003). HA binding to cell-receptors is responsible for supporting cellular activity, health, etc. Indeed, tissue pathogenesis can occur as a result of degraded or defective HA.

Tumor cells over-express receptors of HA and tumor growth and maintenance are supported by binding to HA (Kong, Oncol. Rep. 10:51-55, 2003). Hyaluronidase, a hydrolase enzyme that degrades HA, is secreted by tumor cells and accounts for much of the tissue remodeling capacity of tumor cells. High levels of hyaluronidase, however, have been reported to inhibit tumor cells and disrupt tumor integrity (Zeng, et al., Int. J. Cancer 77:396-401, 1998).

Implants can be significantly more efficient when biomaterial surfaces more closely mimic the conditions and properties encountered in ECM. In some instances, improvements are observed when tissues intended to receive implants are first pre-treated to introduce artificial scaffolds before implantation (Mangano, et al., Int. J. Oral Maxillofac. Implants 18:23-30, 2003). However, a superior approach can be to introduce factors and conditions mimicking the ECM directly on the biomaterial (Segura, et al., Biomaterials 26:1575-1584, 2005). As stem cell technology develops, it will be possible to pre-coat biomaterials with beneficial cells and ECM of patient origin prior to implantation to provide vastly superior biomaterial that will greatly speed the healing process and resist foreign body reactions.

The present invention discloses how to control the composition, concentration, and persistence of healing factors, hyaluronan, and other ECM components on implants and other biomaterials. Additionally, the present disclosure describes how to prevent unwanted non-specific surface fouling on implants and other biomaterials. In combination, these benefits can lead to more natural interfaces of biomaterials in tissues that promote healing and osseointegration and resist negative bodily processes.

In one embodiment, a method of forming a biomolecular coating on a surface of a medical device is disclosed including providing a medical device, where the device comprises one or more gold surfaces and applying a biomaterial to the device, where the biomaterial is adsorbed on or is formed on a surface thereof, and where the biomaterial includes a fusion protein having at least one gold binding protein (GBP) domain and at least one proteinaceous biomolecule domain, where applying the biomaterial immobilizes the biomolecule on the surface, thereby forming a biomolecular coating on the medical device.

Advances have led to development of materials that are compatible with implantation in bone, or osseointegration, providing structural support for a wide array of synthetic devices to replace damaged digits, limbs, joints or teeth. Dental implants are replacement teeth, most commonly based on a titanium screw inserted in the jawbone. The implant process usually requires 2 visits, the first to place the implant and allow bone integration over a period of several weeks, with a follow up visit to attach the crown. Bone growth anchors the screw and provides structural support essentially equivalent to natural teeth. Key factors driving innovation in this field are the push to more rapid or immediate loading (loading refers to usage of the implant) which reduces or eliminates the interval between placement of the implant and completion of the surgery. Furthermore, enhanced osseointegration is important to support stable long-term integration of implants without bone loss.

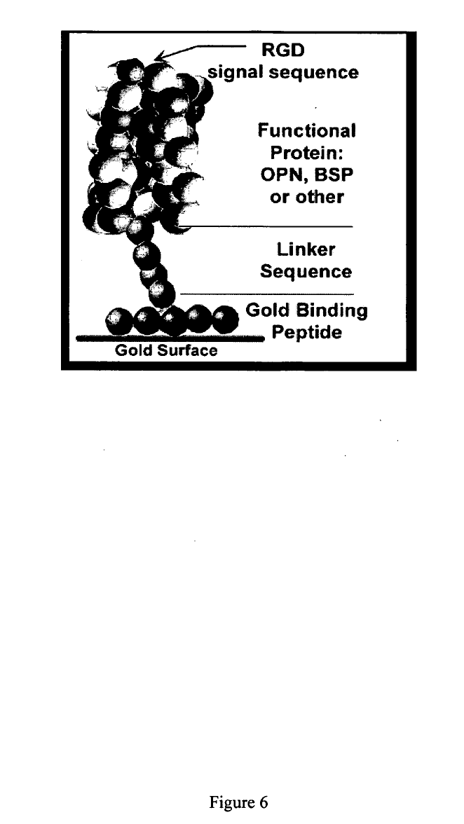

In one aspect, GBP-OPN, GBP-BSP, and GBP-Arg-Gly-Asp-containing peptides are attached to titanium implants that have been coated with gold (see, e.g., FIG. 6). The robust attachment of GBP to gold will provide the fusion partners in an optimum orientation on the surface of the implants, allow interaction of the factors with osteoblasts and other cells at the interface, reduce non-specific binding on the implant surface, and speed up the healing and osseointegration process.

Without the attachment of factors such as BSP, OPN, or Arg-Gly-Asp peptides (Fisher, et al., J. Biol. Chem. 265:2347-2351, 1990; Denhardt and Guo, The FASEB Journal 7:1475-1482, 1993; Rezania and Healy, J. Biomed Res. 52:595-600, 2000; Zriqat, et. al., J. Biomed Mater. Res. A. 64:105-113, 2003) to facilitate healing and osseointegration, orthopedic implants can require many months before they can be used and they often fail. At the very least the presence of appropriate factors can greatly enhance the healing process. Similarly, coronary stents can occlude rapidly, catheters and heart-lung machines can cause thrombosis, electrodes can fail, and heart valves can mineralize if surface fouling and foreign body reactions are not prevented. Even hip and knee replacements only last for 10-20 years. The entire area of biomaterials and implants can be vastly improved by developing surfaces that are more bio-friendly.

For certain applications, e.g., dental and orthopedic implants, pure gold used alone is too soft to provide the mechanical strength needed. However, a thin layer of pure gold can be readily applied to core material, e.g., titanium, to impart the non-fouling and non-cytotoxic properties of gold to implants. Mild allergies to gold, primarily dental gold, have been reported in a small percentage of the population. When follow up studies are conducted, however, the results usually indicate that the allergy is caused by contaminates in the gold preparation or due to another component used in conjunction with gold. Thus, pure gold appears to be extremely safe when used in biomaterials, as one would suspect from the wide-spread use of gold crowns in dentistry.

In a related aspect, the example above for teeth implants can be expanded to any implant that is inserted into bone. Again, titanium can be used as the core material and GBP-OPN, GBP-BSP, and GBP-Arg-Gly-Asp peptide fusion can be attached to a gold layer coating the titanium.

A rapidly developing area promising breakthrough advances in healthcare is the production of artificial organs and tissues for use as replacement materials of diseased or damaged ones. For example, artificial skin is already commercially available and there is much activity in developing artificial hearts, heart valves, blood vessels, bone, fingers, arms, legs, joints, corneas, ligaments, bladders, kidneys, pancreas, adrenal glands, lungs, livers, bone, and many others.

Artificial devices generally are built on scaffolds of biodegradable or biocompatible materials that are designed with shapes, chambers, and other features to mimic the desired organ or tissue. The scaffold is usually constructed with materials including fibrous substances, e.g., silk or collagen, or plastics, silicon, glass, ceramics, polymers, metals, and others (Meinel, et al., J. Biomed. Mater. Res A. 71:25-34, 2004; Meinel, et al., Bone 37:6988-698, 2005; Knabe, et al., Clin. Oral Implants Res. 16:119-127, 2005; Landis, et al., Orthod. Craniofac. Res. 8:303-312, 2005; Young, et al., Tissue Eng. 11: 1599-1610, 2005). Scaffold material is often porous to increase surface area for cell attachment, permit blood vessel formation throughout the device, and to add to the strength of the device.

Such devices typically fail because the body mounts foreign body defenses in attempting to isolate or eliminate the device. If, however, the patient's own organ or tissue specific cells can be integrated with the device there is a significantly higher probability that the body will accept the device as its own. When possible cells, e.g., fibroblasts, are taken from patients and grown in tissue culture with biomaterials to produce an integrated device (Ferrera, et al., Bone 30:718-725, 2002; Zhang, et al., J. Orthop. Res. 22:30-38, 2004). The cells attached to devices can “cross-talk” with interface proteins, macromolecules, and other cells when the device is transplanted into a patient. Since the cells coating the device originate from the patient, there is a high likelihood that the device will be accepted by the body as self and avoid isolation, rejection, and eventual failure. Transplanted devices without a layer of appropriate cells are subject to non-specific surface fouling and foreign body reaction that occurs at the interface of any unprotected surface material and tissue.

The use of efficient artificial organs and tissues will become routine when superior biomaterials are coupled with emerging stem cell technology.

In one embodiment, the application of biomaterials to facilitate the production of artificial organs and tissues that contain healing factors including polypeptides, hyaluronan, and other ECM components is disclosed, including the use of such materials to produce devices that are resistant to foreign body reactions. In combination, such benefits can lead to more natural interfaces of artificial organs in tissues that promote healing and resist negative bodily processes, and which are more likely to succeed when transplanted into patients.

In other embodiments, improvement in the performance of biomaterials transiently implanted or inserted in patients or be exposed to patient's bodily fluids is disclosed. Examples include, urinary catheters, electrodes, tubing or lines connecting patients to kidney dialysis and heart-lung machines, and working surfaces that contact bodily fluids. In these instances, all surfaces exposed to cells, tissues, and bodily fluids are subject to non-specific surface fouling of proteins and cells that can lead to thrombosis, bacterial infection, clogging, and device failure. Current methods used to prevent these surface-induced problems include using low-sticking substances—Teflon or polyethylene glycol on surfaces—adding antibiotics, heparin or introducing other ameliorable substances to a system.

Early coronary stents typically became occluded due to thrombosis and inflammatory processes initiated at the interface of the stent and tissue. The devices frequently required removal and replacement with 6 months. In the past few years, much improvement has been achieved by coating the stents with substances that prevented clot formation and inflammation around the stent. The present invention can be applied to coronary stents to provide even longer-term protection against failure. Stents can be made with gold or other material that is coated with a layer of gold. GBP fusion proteins containing anti-clotting and anti-inflammatory partners can be attached to the stents. In this way the healing factors and the non-fouling property of GBP work synergistically to promote healing and prevent failure of the stent.

In one aspect, uses of the biomaterials as described in the present invention include, but are not limited to, employing nanoparticles in medical applications, such as bone regeneration preparations, bioimaging, drug-delivery, and vaccines.

Gold nanoparticles (GNP) range in size from 1 to 20 nanometer in diameter and can have no surface charge or can have an anionic or cationic surface charge. Larger particles of elemental gold, i.e., gold powder, can be several microns in diameter. Colloidal gold (CAu) prepared from gold salts to form suspended particles (20 nanometer to 100 nanometer in diameter) in aqueous solution typically has an anionic surface charge.

Several lines of evidence indicate that certain “glues” or “cements” consisting of healing factors, small nucleation particles, and a scaffold material for osteoblast attachment will soon be available to replace or regenerate lost bone due to injury or disease (Isogai, et al., Plast. Reconstr. Surg. 105:953-963, 2000; Jin, et al., J. Biomed. Mater. Res. A. 67:54-60, 2003; Ahoa, et al. J. Periodontol 75:154-161, 2004). Ideally, such preparations will contain biocompatible materials, including scaffolds and nucleation particles. Fibrin, collagen, silk, hyaluronan, and synthetic polymers have been investigated as scaffolds (Meinel, et al., J. Biomed. Mater. Res A. 71:25-34, 2004; Meinel, et al., Bone 37:6988-698, 2005; Knabe, et al., Clin. Oral Implants Res. 16:119-127, 2005; Landis, et al., Orthod. Craniofac. Res. 8:303-312, 2005; Young, et al., Tissue Eng. 11:1599-1610, 2005). Nucleation particles used with or without attached healing factors include: porous calcium phosphate, biocompatible glass, ceramics, silicon, polystyrene, other plastics, synthetic porous polymers, titanium, and other metals (Kim, et al., Biomaterial 26:2501-2507, 2005).

The efficacy of these materials in facilitating cell attachment, activation, growth, and proliferation vary considerable and other factors including toxicity can be an issue. For example, titanium one of the safest, generally non-toxic biomaterials can be toxic to cells and also can affect cellular activity below toxic levels when present as small particles or ions (Sun, et al., J. Biomed. Mater. Res. 34:29-37, 1997; Zreiqat, et al., Biomaterials 24:337-346, 2003).

Gold nanoparticles have not been described for use as cell nucleation sites, however, there are many reports attesting to the safety of gold in humans including recent studies evaluating the toxicity of gold nanoparticles when ingested by cells.

A recent study evaluated the use 1.9 nanometer gold particles as a contrast agent in bioimaging and reported its superior properties including non toxicity, long persistence in the blood vessels, little diffusion into tissues, excellent excretion by the kidneys, and high-contrast images (Hainfield, et al., Br. J. Radiol. 79:248-253, 2006).

The results of several studies indicate that gold nanoparticles can be excellent vehicles for drug-delivery or possibly gene therapy because the particles are non-toxic, persistent, can be derivatized with homing molecules to target cells and tissues, and can be charged with bioactive molecules (Yang, et al., Bioconjug. Chem. 16:494-496, 2005; Qin, et al., Langmuir 21:9346-9351, 2005; Rosi, et al., Science 312:1027-1030, 2006).

Small particles in general can improve the efficiency of vaccines for two reasons: first, the particles can act as an adjuvant to stimulate immune processes and; second, natural immunogens, e.g., on viruses and bacteria are, are frequently arranged on surfaces and attachment of immunogens on particles can mimic nature to produce a stronger immune response (Lutsiak, et al., J. Pharm. Pharmacol. 58:739-747; Saupe, et al., Expert. Opin. Drug Delivery 3:345-354, 2006).

Attachment of proteins and other bioactive molecules to the surface of biomaterials can provide certain benefits when applied in vivo that are not possible using the same amount of bioactive molecules in solution. For example, immobilization can result in significant persistence of bioactive molecules at interfaces compared to soluble molecules in blood or other body fluids or tissues that are subject to rapid clearance from the body. Also, immobilization of bioactive molecules on surfaces can impart significant resistance to hydrolytic enzymes and other destructive processes that freely soluble molecules can be susceptible to in bodily fluids and tissues. Additionally, in the special case of nanoparticles derivatized with bioactive molecules, cellular uptake can occur, thereby, offering an efficient method to introduce bioactive molecules into cells, e.g., cancer cells, or transport molecules into, through, and out again of cells, e.g., epithelial or endothelial cells. The later process can provide a method to transport derivatized nanoparticles across cellular membranes, e.g., from blood to tissues. Gold nanoparticles can be especially efficient in such applications targeting cellular transport of bioactive molecules because the ingested particles are not toxic to cells and the particles can have long-half lives in body fluids and cells.

Pure gold is strongly attracted to certain sulfur-containing compounds such as sulfides and thiols (also called sullhydryls) under appropriate conditions. A widely used method to establish a foundation layer on gold employs alkanethiols-typically 10-15 carbons long—that can form a self-assembling monolayer (SAM) when the sulfur associates with gold (Bain, et al., J. Am. Chem. Soc. 111:321-335, 1989; Lofas and Johnsson, J. Am Chem. Soc. Commun 117:12528-12536, 1990). Interaction of adjacent alkyl chains can stabilize the SAM. Reactive groups, e.g., amino or carboxyl groups positioned at the distil end of the alkanethiols can be used to attach bioactive or other molecules (Johnsson, et al., BioTechniques 11:620-627, 1991).

SAMs consisting of alkanethiols can be durable on gold for applications performed under highly controlled, well-defined laboratory conditions. SAMs can be unstable, however, when application conditions are variable or extreme. For example, even the most stable alkanethiol SAMs on gold begin breaking down at approximately 50° C. and “melt” at 75° C. (Pradeep and Sandhyarani, Pure Appl Chem 74:1593-1607, 2002). Also, sulfides, thiols, and other sulfur reactive compounds often present in biological fluids and environmental solutions can disrupt the integrity of SAMs on gold. Complex solutions such as blood or environmental samples contain many proteins and other substances that contain sulfur, e.g., cysteinyl residues and disulfide bonds that can displace alkanethiol SAMs on gold.

Covalent attachment chemistries are available for the linking of protein to surfaces, based on reactivity of specific amino acids (e.g., lysine, glutamate, histidine and others) or on the amino or carboxy termini. Frequently, a reactive foundation layer must be introduced on the surface to attach proteins. Foundation layers may introduce additional problems, such as durability, background interference, and decreased electrode conductivity. The idiosyncratic nature of enzyme properties precludes general application, since the use of a specific chemical method can produce variable success for different proteins. In addition, where chemistry is dependent on modification of specific amino acids, the chemistry itself may destroy enzyme activity. Further, coupling reactions can require harsh solvent or extreme conditions that may inactivate enzymes or adversely affect cofactors.

Affinity capture methods have been developed using surface attached proteins such as Streptavidin/Avidin to bind enzyme-biotin conjugates. This approach can provide stable attached enzymes, but attachment of Streptavidin directly to surfaces or to foundation layers has the same constraints as described above.

Peptides derived from adhesive proteins in marine mussels that have been derivatized with modified polyethylene glycols (PEG) are being developed into fouling-resistant compounds for biomaterials. Stable attachment to a variety of materials, including gold, is facilitated by the cross-linking of adjacent molecules via 3,4-dihydrophenylalanine (DOPA) amino acid residues contained in the mussel peptides (Dalsin, et al., J. Am. Chem. Soc. 125:4253-4258, 2003; Hwang, et al., Appl. and Environ. Microbiol 70:3352-3359, 2004; Startz, et al., J. Am. Chem. Soc. 127:7972-7973). The anti-fouling property is due to the PEG component. The entire process to generate fouling-resistant surfaces requires several separate chemical steps, unlike the present invention which is a one-step process completed in a few minutes. The strength of mussel adhesive peptide binding to surfaces is a result a molecular cross-linking mechanism (Hwang, et al., Appl. and Environ. Microbiol 70:3352-3359, 2004). The long-term avidity of mussel adhesive peptide binding to gold and the question of toxicity of the compound in vivo has yet to be investigated.

To date, the mussel adhesive and similar peptides derivatized with PEG have been used to prevent surface fouling. While this goal is important in developing implants, other biomaterials, and biodetection platforms, it is equally important to attach factors such as BSP and OPN to facilitate healing and osseointegration or biodetection molecules to surfaces. The present invention discloses that GBP technology can be used to achieve resistance to non-specific surface fouling and, simultaneously, derivatize surfaces with bioactive molecules.

In contrast to other linking means, no linking chemistry is required to attach desired polypeptides to GBP. With conventional methods different coupling chemistries can be required to attach distinct proteins to a GBP or other foundation layer. For example, when protein array chips are constructed with hundreds or thousands of unique proteins the complexity of many different linking chemistries, variable reaction rates and unequal protein coupling present formidable challenges to achieve functional uniformity on any single array and consistency among replicate arrays. The recombinant molecules provided by the present invention eliminate these technical difficulties and uncertainties by simplifying the entire surface derivatization process to a single, rapid step, i.e., the specific interaction of GBP and gold. Thus, in a related aspect, a method is provided to achieve high uniformity and consistency in the manufacture of gold chips, colloidal gold, or any gold surface consisting of one or many distinct recognition or binding polypeptides or enzymes.

In one embodiment, the invention encodes a gold-binding peptide (GBP) for the stable attachment of fusion proteins to any gold surface. In a related aspect, a second component includes, but is not limited to, a fusion partner consisting of any desired polypeptide with specific binding or enzyme activity. For example, the inclusion of short, flexible amino acid sequences of low complexity linking GBP and fusion partner domains facilitates optimum physical orientation of each domain to allow full expression of GBP and fusion partner activities. In another related aspect, a third component including, but not limited to, a specific polypeptide affinity tag, e.g., polyhistidine (His₆-tag), permits rapid purification of the fusion protein in essentially one step. Rapid purification from cellular extracts or secretions can minimize proteolytic degradation typically associated with the expression of fusion proteins. In one aspect, the presence of the affinity tag in fusion proteins, obviates the need for each fusion protein to require a separate purification scheme.

In another aspect, the disclosed method allows for the attachment of proteins and small polypeptides to gold by transferring the gold-binding process to a polypeptide domain designed for this purpose (i.e., GBP). Further, the invention provides a rapid, one-step purification procedure that can be used for all fusion proteins of the type disclosed.

In one aspect, such fusion proteins include, but are not limited to, specific chemical or enzyme cleavage sites in the linking amino acid sequences between domains to allow the physical separation of fusion partner domains.

In one embodiment, the invention provides for GBP fusion proteins comprising one or thermophilic or extremophilic enzymes. In a related aspect, “thermophilic” is used to identify enzymes which resist destabilization of domain structure due to exposure to temperature ranges that would normally denature equivalent mesophilic enzymes (e.g., temperatures in the range of 40° C. to 100° C.). In another related aspect, “extremophilic” is used to identify enzymes which resist destabilization of domain structure due to exposure to temperature ranges and/or chemical conditions that exceed temperature and/or ordinary chemical conditions which are used to define equivalent mesophilic enzymes. In contrast, a mesophilic enzyme would function best at moderate temperatures (e.g., between 25° C. and 40° C.), moderate pH environments (e.g., 7.0-7.5), or under moderate ionic strength (i.e., ionic strength does not effect the relative total net charge of the enzyme such that the distribution of charge on the exterior surface of the enzyme destabilizes the function of catalytically active groups).

A list of such thermophilic and extremophilic enzymes, which is by no means exhaustive, is provided in Tables 1 and 2 below.

TABLE 1
Thermophilic enzymes

| Enzyme | Source | Application | Accession No. |
|---|---|---|---|
| Taq Polymerase | Thermus aquaticus | PCR technologies | AAD44403 |
| Deep Vent DNA | Pyrococcus species | ″ | CAJ90576 |
| polymerase | | | |
| Pfu DNA ligase | Pyrococcus furiosus | Ligase chain reaction and | P56709 |
| | | DNA ligations | |
| Serine protease | Thermus thermophilus | DNA and RNA | YP_004973 |
| | HB27 | purifications; cellular | |
| | | structures degradation | |
| | | prior to PCR | |
| Methionine | Pyrococcus horikoshii | Cleavage of N-terminal | NP_142587 |
| aminopeptidase | OT3 | Met in proteins | |
| Carboxypeptidase | Sulfolobus solfataricus | C-terminal sequencing | P80092 |
| Alkaline phosphatase | Geobacillus kaustophilus | Diagnostics: enzyme | BAD76986 |
| | HTA426 | labeling application where | |
| | | high stability is required | |
| BstXI | Geobacillus | Restriction endonuclease | AAN03687 |
| | stearothermophilus | | |
| Kpn2I | Klebsiella pneumoniae | Site specific DNA | CAC41108 |
| | | methyltransferase | |
| TaqI | Thermus aquaticus | Type II restriction enzyme | P14386 |
| Tsp451 | Thermophilis sp. | Restriction endonuclease | O51936 |

TABLE 2
Extremophilic enzymes

| Enzyme | Source | Application | Accession No. |
|---|---|---|---|
| Endo-1,4-β-glucanase | Thermotoga neapolitana | Cellulose degradation | CAA8808 |
| Cellobiohydrolase | Chaetomium | ″ | AAW64926 |
| | thermophilium | | |
| Endoxylanase | Cellulomonas fimi | Paper pulp bleaching | CAA90745 |
| β-xylosidase | Oceanobacillus iheyensis | ″ | NC_004193 |
| | HTE831 | | |
| β-mannanase | Caldicellulosiruptor | Softwood pulp bleaching | P22533 |
| | saccharolyticus | | |
| β-glucosidase | Thermococcus sp. | Regio- and stereoselective | CAA94187 |
| | | glucoconjugate synthesis | |
| | | by transglycosylation | |
| Trehalose synthase | Sulfolobus shibatae | Used in food, cosmetics, | AAM8159 |
| | | medicine, and organ | |
| | | preservation | |
| Hydantoinase | Aeropyrum penix K1 | Synthesis of D-amino | NP_148671 |
| | | acids as intermediates in | |
| | | the production of semi- | |
| | | synthetic antibiotics, | |
| | | peptide hormones, | |
| | | pyethroids, and pesticides | |
| Esterase | Geobacillus | Transesterification and | BAA02182 |
| | stearothermophilus | ester synthesis | |
| Aldolase | Geobacillus kaustophilus | Synthetic chemistry, C—C | YP_146808 |
| | HTA426 | bond synthesis | |
| Cytochrome P450 | Sulfolobus solfataricus | Selective region- and | Q55080 |
| | | stereospecific | |
| | | hydroxylations in chemical | |
| | | synthesis | |
| Secondary Alcohol | Clostridium biejerinckii | Chemical synthesis: | 1PEDA, 1PEDB, 1PEDC, |
| dehydrogenase (Chains A-D) | | production of | 1PEDD |
| | | enatiomerically pure chiral | |
| | | alcohols | |
| Pectate lyase | Clostridium stercorarium | Fruit juice clarification, | BAC87940 |
| | | wine making, fruit and | |
| | | vegetable maceration | |
| β-galactosidase | Thermoanaerobacter | Production of lactose free | CAC50570 |
| | mathranii | dietary milk products | |
| Phytase | Talaromyces thermophilus | Phytate degradation in | AAB96873 |
| | | animal feed | |
| Chitinase | Thermomycetes | Chitin utilization as a | AAY99632 |
| | lanuginuosus | renewable resource; | |
| | | production of biologically | |
| | | active oligosaccharides | |

In one aspect, biosensors are disclosed to monitor industrial, bioremediation, and other processes on-line under prevailing high temperatures rather than sampling and cooling solutions to make analysis possible. Real-time biosensing throughout the entire process saves time, effort, money, and may be a more reliable indication of conditions. Continuous monitoring of ongoing processes can signal precise times to start or end important protocol steps.

Many thermophilic enzymes, polypeptides, lipids, and other bioactive molecules are not only stable at high temperature, but they are also more active in harsh chemical agents and water-miscible organic solvents than their mesophilic counterparts (Lasa and Berenguer, Microbiologia 1993, 9:77-89). Therefore, in another aspect, biosensors are disclosed that can function in extreme chemical environments encountered, e.g., in industrial processes, environmental monitoring, bioremediation, and chemical reactors. As stated above for processes at high temperature, biosensors capable of continuous monitoring of processes requiring harsh or extreme chemical environments can be beneficial.

In one aspect, biosensors are disclosed that function in extreme chemical environments, e.g., in industrial processes, environmental monitoring, bioremediation, and chemical reactors. As in the above example for processes at high temperature, biosensors capable of continuous monitoring of processes requiring harsh or extreme chemical environments can be beneficial.

In other aspects, derivatization of gold with GBP-fusion proteins containing thermophilic or extremophilic enzymes and other bioactive molecules are disclosed including, but not limited to, Taq polymerase, thermophilic nucleic acid restriction enzymes, heat shock or chaperone proteins, thermophilic proteases (e.g., thermolysin), and catalases.

Very little is known about the biochemistry and cellular mechanisms of thermophilic organisms other than they are significantly different from those in mesophilic organisms. Unique, essentially unknown, mechanisms operate at extreme temperatures to keep cell membranes intact, allow cellular processes, and to support DNA replication and protein synthesis. Biosensors can be extremely beneficial devices to study thermophilic biochemistry in real-time, especially processes involving bi or multi molecular interactions. Therefore, in addition to the commercial applications described above that can benefit from real-time monitoring, the present invention can provide novel biosensors capable of operating at high temperatures for investigating the biochemical and cellular mechanisms of thermophiles at extreme temperatures. This will be a significant advance in the field. Without limiting the scope of the invention, examples of biosensors described in the present invention that have potential to operate at high temperatures include enzyme electrodes, piezoelectric quartz crystals, surface plasmon resonance, and DNA and protein micro arrays.

In another aspect, the present invention also discloses non-sensing devices and materials, e.g., lab-on-a-chip platforms, biomedical devices, and biomaterials using thermophilic biomolecules attached to gold that can benefit research and healthcare.

In a related aspect, the present invention discloses biosensors, microarrays, and other devices for specific applications utilizing non-thermophilic extremophilic biomolecules. For example, devices can be constructed to operate in highly acidic, concentrated sulfur-containing, or high salt environments, such applications that cannot be supported by mesophile molecular analogues.

In one aspect, GBP-fusion proteins are used to provide a durable GBP layer on biomaterials having a gold surface and implanted or injected into patients that are resistant to fouling by blood and tissue proteins, other macromolecules, cells, tissues, and bacteria.

In another aspect, the molecular orientation and surface presentation of a ligand contained in GBP fusion proteins can be controlled to provide the optimum binding to specific cell receptors. Those skilled in the art recognize that healing, growth, and other beneficial factors attached to an implant surface will interface most optimally with target cells when the factors have freedom of movement to best interact with specific cell-surface receptors. Physical adsorption and chemical attachment of factors to a surface are typically random processes resulting in many non-productive molecules on surfaces. The present invention provides a method that ensures surface attached factors will have the freedom of movement to interact productively with cell-surface receptors. The GBP fusion proteins are designed to permit individual domains to perform independently of each other by inserting flexible linkers consisting of repeating Gly-Ser sequences of various length. Therefore, gold binding occurs through GBP and the bioactive fusion partner is tethered off the surface into the interface solution where it can effectively bind cell-surface receptors.

In many instances there is an optimum density of growth and other factors that facilitates robust interfacing of biomaterials and cells/tissues. High surface density can adversely affect the attachment, growth, proliferation, and activity of attached cells. The present invention can be applied to control the surface density of beneficial factors on biomaterials. For example, the density of GBP-OPN, GBP-BSP, and GBP-Arg-Gly-Asp peptide fusions on gold coated implants can be controlled by adding appropriate amounts of GBP to the fusion proteins prior to the gold binding step.

In another aspect, various mixtures of GBP and GBP fusion proteins containing healing factors can be used to coat biomaterials to achieve an optimum level of resistance to surface fouling, healing, and avoidance of negative effects that excessively high concentrations of “healing factors” can have.

In another aspect, controlled layering of gold on implants and other biomaterials can achieve a patterned surface that can enhance desired cell adhesion, proliferation and activity.

In other aspects, components of extracellular matrix (ECM), including collagen, fibronectin, hyaluronic acid, and proteoglycans can be attached to gold to provide a 3-dimensional surface environment that can significantly improve “cross-talk” with cells at interfaces of implants.

In another aspect, gold layering of scaffold material used in producing artificial organs and tissues can be coupled with GBP to develop devices. The present invention can be applied to the field of artificial organs and tissues when scaffolds are coated with a layer of gold. Those skilled in the art can establish gold coatings on scaffolds by chemical methods (Delvaux, et al. Biosensors & Bioelectronics 20:1587-1594, 2005). Such chemical processes are ideal for coating intricate surfaces of porous materials frequently used for scaffolds. GBP fusion proteins containing OPN, BSP, and Arg-Gly-Asp peptides can then be attached to the scaffold to facilitate osteocyte and other cell attachment in tissue culture. In this manner, artificial segments of bone could be produced for grafting into a patient to replace lost bone.

Similarly, other growth factors can be fused to GBP and attached to gold coated scaffolds for producing other artificial organs.

In other aspect, tubings, catheters, and operating parts of medical devices exposed to body fluids can be protected against surface fouling, bacteria infection, and blood clot formation. The surfaces of the linings of tubes, catheters, and connections attached to various medical devices are prone to clogging and infection caused by blood components and bacteria. Blood clotting is a major problem. Conventional approaches to prevent blood clotting and infection in these connections include the addition of heparin and antibiotics. The present invention can be applied to produce superior linings of tubes, catheters and connectors that resist blood clotting and infection. Those skilled in the art can coat the interior of tubing material with gold using chemical methods. GBP-fusion proteins containing anti-clotting and antibiotic peptides can be attached to the gold. The combination of the anti-fouling property of GBP and therapeutic factors can provide better connections to medical devices.

In other aspects, gold nanoparticles are coated with factors to stimulate bone mineralization can be used to facilitate healing of fractured bones in older patients and restore lost bone tissue due to disease or surgery.

In other aspects, factors attached to gold coated biomaterials as GBP fusion proteins can be released in tissues over time when desired.

In another aspect, GBP/gold complexes can be used as drug-delivery systems that target specific cells, tissues, and organs when injected into patients. Gold nanoparticles appear safe when injected into animals (Yang, et al., Bioconjug. Chem. 16:494-496, 2005; Qin, et al., Langmuir 21:9346-9351, 2005). GBP fusion proteins containing Arg-Gly-Asp peptides can be attached to nanoparticle gold and used to target and disrupt cancer cells that over express cell-surface integrin receptors. In another example, many types of cancer cells over express the cell surface transferrin receptor. Gold nanoparticles coated with GBP-transferrin fusion protein and, also, containing anti-cancer drugs can be effective in killing certain cancer cells.

In another aspect, GBP/gold complexes are used as contrast agents for bioimaging of tumors, tissues and organs. Nanoparticle gold appears to be a superior contrast agent for bioimaging. The gold persists longer than conventional agents, does not accumulate in tissues, is effectively excreted by the kidneys, is nontoxic, and provides superior images (Qin, et al., Langmuir 21:9346-9351, 2005). The present invention can be used to enhance the contrast agent property of nanogold when GBP-fusion proteins containing tissue or organ specific recognition molecules are attached to the gold particles. In this manner, the particles can accumulate and persist longer in targeted tissues and organs to enhance bioimaging.

In another aspect, GBP/gold complexes can be used as adjuvants in vaccines. Gold nanoparticles can be used effectively in vaccines by providing two important processes. First, the gold particles when injected can serve as an adjuvant or irritant to facilitate immune processes. Second, the display of immunogens on a surface appears to mimic how the body “sees” foreign proteins on invaders. This is particularly true for virus proteins. The present invention can be used to produce more effective vaccines by attaching GBP-fusion proteins containing immunogenic partners to nanoparticle gold.

In one aspect, fusion partners can be attached at either end of the GBP domain. Thus, methods are disclosed which permit two or more copies of a desired fusion partner attached to a single GBP domain to increase the specific binding capacity or enzymatic activity of the fusion protein attached to gold. For example, multiple copies of fusion partners can be expressed in tandem. In a related aspect, a minimum of two copies of a fusion partner can be expressed by placing one at the amino-terminus and the other at the carboxy-terminus of a single GBP domain.

In one embodiment, a method of producing fusion proteins containing two or more distinct fusion partners with different activities is disclosed. For example, a chimera can be produced containing streptavidin at one end of GBP and OPN or BSP at the other end. In a related aspect, a fusion protein with multiple function is one containing two distinct proteinaceous domains attached to GBP. In another aspect, a mixed-function fusion protein is one whereby one fusion partner, e.g., a single-chain antibody or receptor, can bind specific molecules present in low concentration. The increased concentration of specific molecules in the vicinity of the fusion protein can significantly improve the activity of a second fusion partner, e.g., an enzyme that utilizes the specific molecules as substrate when conditions are changed to release the specific molecules from the binding domain of the fusion protein.

In another aspect, recombinant Streptavidin-GBP fusion is 5- to 10-fold more active in binding biotinylated molecules than is recombinant Streptavidin lacking the GBP domain when each are bound to gold.

In one aspect, there is no requirement to purify GBP or the desired protein prior to adsorbing them onto gold. The affinity and specificity of GBP to gold are sufficiently high, e.g., KD=1.5×10⁻¹⁰M to allow specific interaction in crude preparations containing many irrelevant proteins and other macromolecules.

The one to one relationship of GBP to fusion partner in the recombinant molecules enables the construction of uniform foundation layers containing high densities of functional protein. This can increase the sensitivity of detection in applications compared to that provided by conventional chemical attachment methods.

In a related aspect, the recombinant molecules can be constructed to orient recognition proteins appropriately to position their active sites outward from the gold surface to provide optimal interaction with target or substrate molecules. This is accomplished by placing the GBP domain at the N-, or C-termini, or within a surface loop of the recognition protein with linkers consisting of flexible amino acid sequences between domains. Conventional chemical attachments to GBP (Woodbury, et al., Sensors & Bioelectronics, 13:1117-1126, 1998) or other layers typically do not produce proper orientation to permit complete accessibility to binding sites on recognition proteins.

Expression plasmids disclosed herein can be readily adapted for the production of virtually any polypeptide. Once the expression hosts are created, unlimited quantities of many different GBP-containing recombinant proteins can be produced to create, for example, diverse arrays of proteins to facilitate proteomic research and drug screening. The gold-binding process is facilitated by the GBP domain common to each recombinant protein, thereby, ensuring attachment of all desired polypeptides, regardless of intrinsic, or lack of, attraction of the fusion partner to gold. Further, the one to one relationship of GBP and its fusion partner allows the attachment to gold of equimolar amounts of hundreds or thousands of distinct recombinant molecules with different binding or enzyme activities. These benefits derived from the invention, herein, will significantly enhance the construction and performance of protein arrays, nanotechnology-based devices and the like.

The molecular approach described, herein, provides methods for introducing significant improvements in introducing a variety of functions to gold surfaces not possible by existing technology. For example, genetic engineering can produce a recombinant molecule containing GBP and the smallest possible form of a recognition protein that retains binding specificity. This provides at least three benefits. First, reduction of a protein to its specific binding domain eliminates other domains that may contribute complicating allosteric binding events or that could add to background interference. Second, in general, small functioning proteins are less susceptible than larger ones to proteolytic degradation when exposed to biologic fluids. Third, in the example of certain biosensing instruments, binding events occurring nearer the sensing surface produce stronger signals than those occurring farther away from the surface. Thus, the smaller the recognition protein, the higher the sensitivity of detection. A further benefit of the molecular approach is that appropriate modifications can be introduced into the protein sequence to produce a recombinant molecule with increased stability or other improvements. For example, if a region of the recombinant molecule is susceptible to proteolysis, introducing appropriate amino acid substitutions in the fusion protein may prevent degradation.

GBP fusion proteins can be arranged in several different ways. The GBP sequence can be positioned at the amino terminus, internally or at the carboxyl terminus. DNA sequences encoding the fusion protein portion of plasmid vectors can be expressed in bacterial, baculoviral, yeast, plant or mammalian cell hosts.

In this disclosure, detailed methods for expressing GBP-based fusion proteins, rapid purification, characterization of activities, and specific examples for applications are described, including homo- or heterodimers or higher complexes of proteins and macromolecules required for a specific biologic function.

The present invention describes the fabrication of superior colloidal gold (CG)- or nanogold (NG)-polypeptide complexes compared to conventional methods. Bioactive polypeptides are fused to GBP to allow binding of polypeptides to CG, NG, or any type of gold-coated beads or particles.

In one embodiment, methods are disclosed for expressing and producing GBP-fusion proteins that contain bioactive polypeptides for the purpose of immobilizing the bioactivity on CG or NG. This technology has the potential of delivering any desired polypeptide directly to CG or NG regardless of the polypeptides intrinsic gold-binding capacity. It eliminates the use of inefficient or activity-destroying attachment methods and it provides reproducible stability. GBP optimally binds gold at pH 7 to 8, which is an ideal range for retention of bioactivity for most polypeptides. The 1:1 correspondence between the gold-binding and the bioactive polypeptide structures allows high-density surface binding. With optimum positioning of the GBP element, polypeptides can be tethered on surfaces to express full activity in the surrounding solution. In contrast, physical adsorption and chemical coupling methods can lead to surface denaturation and inactivation of polypeptides, and non-productive binding. The approach described herein provides high attachment efficiency, fidelity, and retention of activity that can lead to the development of more robust and sensitive forms of derivatized CG or NG.

Relatively few naturally occurring proteins bind strongly to CG or NG using standard procedures or retain full bioactivity when binding does occur. The presence of salt can prevent protein binding to gold. Many proteins are insoluble or bind other surfaces in low salt concentrations. Also, protein binding to CG or NG is favored at a pH close to the pI of the molecule. But many proteins of interest have reduced solubility near their pIs. Importantly, few small peptides of interest bind CG or NG directly and, therefore, many potential clinical and other testing applications are not possible using conventional methods. In a related aspect, the methods disclosed allow for gold binding of any fusion polypeptide to the GBP domain regardless of the intrinsic binding affinity of its partner and under conditions, i.e., pH 7 and moderate salt concentration that favor retention of activity and solubility of polypeptides. Further, the use of significantly less protein to saturate gold surfaces is observed because binding is facilitated and accelerated through GBP.

The methods and compositions disclosed allow for facile production of various iterations of CG and NG with GBP-fusion proteins containing bioactive polypeptides. Further, the invention allows for the use of small particles such as latex beads, plastic beads, or the like that have been coated with thin layers of gold to which GBP-fusion proteins containing bioactive polypeptides can be attached. The advantages of using gold-coated particles include, but are not limited to lower cost, more readily produced materials, easier to use materials, improved testing properties, greater stability during storage and testing, and wider application potential compared to existing methods.

In another aspect, medical devices comprised of non-gold materials can be coated with a thin layer of gold without altering the basic electrical, physical, or mechanical properties of the substrate material. GBP-fusion proteins can then be added to the surface to provide biological activity or a biocompatible film or protective barrier.

In another aspect, micro-array chips and other devices comprised of non-gold materials can be coated with a thin layer of gold without altering the basic chemical, electrical, or physical properties of the underlying substrate material. GBP-fusion proteins can then be added to the surface to provide biological activity.

In another aspect, bioimaging or biocontrast agents comprised of non-gold materials can benefit using GBP-fusion proteins by coating the agents with a thin layer of gold.

In another aspect, therapeutic materials including, but not limited to, radioactive or other cytotoxic metals or other cytotoxic materials can be coated with a thin bioprotective layer of gold; derivatized with GBP-fusion proteins containing specific antibodies, or cell receptor ligands, or other cell specific binding molecule, or other tissue specific binding molecule; and the derivatized material can be targeted and concentrated on or in specific cells, tissues, or organs, or cancerous tumors.

In another aspect, a fusion protein consisting of GBP and tissue elastin can be bound to a biosensing device to measure elastase activity in tissue extracts, or cell extracts, or body fluids, or cell culture medium.

In another aspect, a fusion protein consisting of GBP and fibrin can be bound to a biosensing device to measure fibrinolytic activity in tissue extracts, or cell extracts, or body fluids, or cell culture medium.

In another aspect, a fusion protein consisting of GBP and any of a variety of blood coagulation factors can be bound to a biosensing device to measure the specific activity of factor activation in tissue extracts, or cell extracts, or body fluids, or cell culture medium.

In another aspect, a fusion protein consisting of GBP and any of a variety of blood complement proteins can be bound to a biosensing device to measure the specific activity of protein activation in tissue extracts, or cell extracts, or body fluids, or cell culture medium.

In another aspect, a fusion protein consisting of GBP and any of a variety of proteins involved in the process of apoptosis can be bound to a biosensing device to measure the specific protein activation activity in cell extracts or cell culture medium.

In another aspect, a fusion protein consisting of GBP and a specific polypeptide substrate of a protease on or secreted from cells can be bound to a biosensing device to measure the specific protease activity on cells, or in cell extracts, or secreted by cells into culture medium or body fluids.

In another aspect, a fusion protein consisting of GBP and a specific polypeptide substrate of a protease required for viral processing can be bound to a biosensing device to measure the specific protease activity in tissue extracts, or cell extracts, or body fluids, or in cell culture medium.

In another aspect, a fusion protein consisting of GBP and a specific polypeptide substrate of a protease secreted from or residing on a parasite can be bound to a biosensing device to measure the specific protease activity in tissue extracts, or cell extracts or body fluids, or in cell culture medium.

In many other aspects, a fusion protein consisting of GBP and a specific polypeptide inhibitor(s) of a protease can be bound to a biosensing device to detect the presence of a protease in test samples. The device can be used to quantify protease levels in tissue extracts, plant extracts, parasite extracts, cell extracts, body fluids, or in cell culture medium.

The following examples are intended to illustrate but not limit the invention.

### EXAMPLES

### Example 1

Plasmid Design for Expression of GBP Fusion Proteins

Recombinant fusion proteins are produced by expression of plasmid constructs encoding the protein of interest fused with the GBP. The plasmid constructs include a selectable marker including but not limited to ampicillin resistance, kanamycin resistance, neomycin resistance or other selectable markers. Transcription of the GBP fusion protein is driven by a regulatable promoter specific for expression in bacteria, yeast, insect cells or mammalian cells. The construct includes a leader sequence for expression in the periplasmic space, for secretion in the media, or for secretion in yeast or mammalian cells or insect cells. Plasmid constructs include multiple cloning sites for insertion of protein sequences in frame with respect to the GBP polypeptide. The GBP sequence can be inserted at the amino-terminal or C-terminal end of fusion partners or inserted between the coding sequence of one or more fusion partners. More than one GBP domain can be fused to a single fusion partner. More than one fusion partner can be fused to a single GBP sequence.

Herein described is the design of a modular set of vectors to support the production of amino and carboxyl terminal fusion proteins in E. coli expression systems. Included are the addition of amino or carboxy affinity tags for purification; the addition of flexible linking sequences between domains to provide independent activity of fusion partners; the presence of a specific cleavage site to disconnect fusion partners if desired; and the requirement for highly regulated expression where toxicity of the over-expressed fusion protein could limit production.

General Methods:

Media. Strains and Transformation: LB media (Bacto L B broth, Miller, from Difco) was used as the basic growth media throughout the course of this study. The antibiotic ampicillin was used at a concentration of 150 μg/ml on plates and at 100 μg/ml in liquid media for the selection and growth of plasmid containing cells. NovaBlue cells from Novagen served as the E. coli host for transformation and expression. Transformations were performed according to the manufacturer's protocol.

Molecular Biology Supplies: All restriction endonucleases and T4 DNA ligase were purchased from New England Biolabs and the kit for DNA sequencing for the Big Dye terminator cycle sequencing from PE/ABI. Plasmid DNAs were made using the miniprep plasmid kits from Qiagen and DNA was extracted from agarose gel slices with is gel extraction kits from either Qiagen or Eppendorf. All reagents were used according to the manufactures' protocols.

Construction of the Expression Plasmid for OPN-GBP Fusion Protein.

The plasmid pSB3053 obtained from S. Brown (Brown, Nat. Biotechnol. 15:269-272, 1997) was used as the source of the GBP fragment containing seven repeats of the peptide MHGKTQATSGTIQS (SEQ ID NO:17). Upon DNA sequencing it was found that the last repeat carried a substitution of the threonine residue in the fifth position for an isoleucine. All the fusion proteins constructed in this work have this substitution.

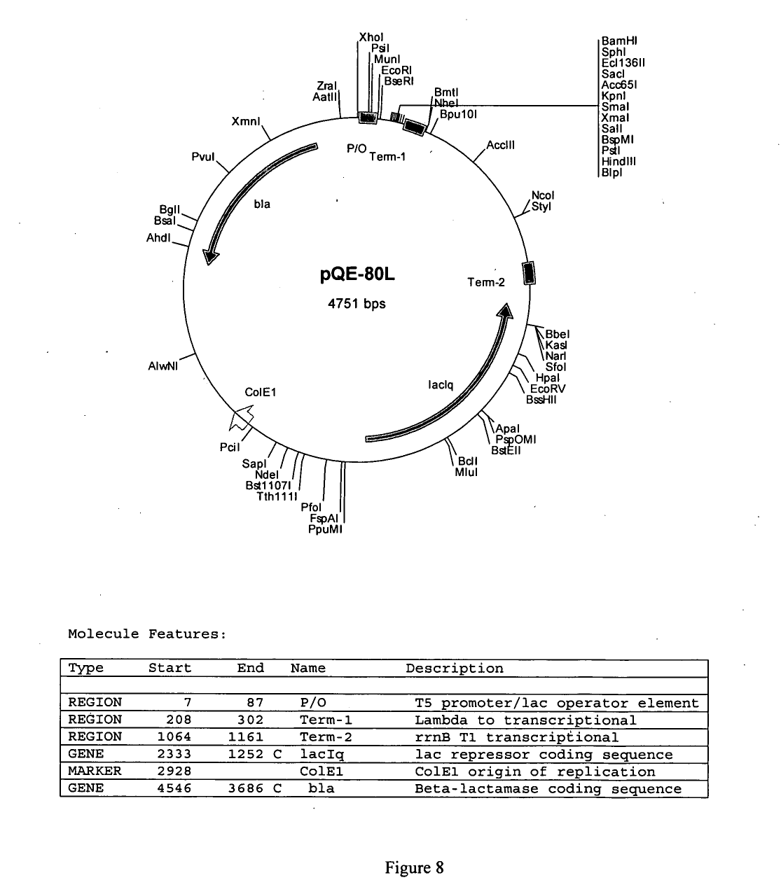

An EcoR I-Xho I fragment encompassing the GBP coding sequence was excised from pSB3053 and adapted at the 3′ end to include coding triplets for the amino acids EGP and a stop codon. Oligonucleotides BH3 (5′ TCG AGG GTC CGT AAT A 3′: SEQ ID NO:18) and BH4 (5′ AGC TTA TTA CGG ACC C 3′: SEQ ID NO:19) were annealed to obtain an adaptor with Xho I and Hind III cohesive ends. The EcoRI-Xho I GBP containing fragment and the adaptor were assembled in pUC 18 and cut with EcoR I and Hind III in a three-part ligation to obtain plasmid pBHI-1. The Bsl I-Hind III fragment from pBHI-1 carrying the GBP coding sequence was adapted at its 5′ end to include an in-frame linker sequence with an Asn-Gly hydroxylamine sensitive cleavage site. Oligonucleotides BH1 (5′ CTG GTA GTG GCA ATG GTC ATA TGC 3′: SEQ ID NO:20) and BH2 (5′ TAT GAC CAT TGC CAC TAC CAG AGC T 3′: SEQ ID NO:21) were annealed to obtain an adaptor with Sac I and Bsl I cohesive ends. The adaptor also incorporates an Nde I site at the methionine codon of the first GBP repeat for ease of adaptation of the GBP fragment with any desired in-frame sequence. Plasmid pBHI-2 was generated with the Bsl I GBP fragment this adaptor and pUC19 linearized with Sac I and Hind III, in a three-part ligation. The nucleotide sequence of the Sac I-Hind III, double-adapted GBP fragment was confirmed by DNA sequencing. Amino acids residues 17-300 of human OPN (Young et al., Genomics 7:491-502, 1990) were used. The source was a synthetic DNA codon optimized for E. coli expression encoding OPN and a short spacer sequence. The final expression plasmid for the His₆ tagged OPN-GBP fusion protein was constructed by ligating the synthetic DNA fragment (BamHI-SacI) and the SacI-HindIII fragment from pBHI-2 into pQE-80L (FIG. 8, Qiagen, Inc.) cut with BamHI and HindIII to obtain an in-frame fusion. The nucleotide sequence of the encoded fusion protein was confirmed by DNA sequencing.

Construction of the Expression Plasmid for BSP-GBP Fusion Protein.

A strategy similar to the one described for the OPN-GBP fusion was used. Amino acid residues 17-317 of human BSP were employed (Fisher et al., J Biochem 265:2347-2351, 1990). The nucleotide sequence of the His₆ tagged BSP-GBP fusion protein in the resulting expression plasmid pBSP-GBP was confirmed by DNA sequencing.

Synthetic DNA encoding core-streptavidin amino acid residues 13-133 (Sano et al., J Biochem 270:28204-28209, 1995) was used to build the expression vector pBHI-28 for expression of a His₆ tagged fusion protein ending with the residues SSSSLIS. The vector pQE-80L (FIG. 8, Qiagen, Inc.) was employed as the backbone expression plasmid.

A plasmid pBHI-29 was also built in a similar fashion to express the His₆ tagged fusion protein streptavidin-GBP.

The expression constructs contain DNA that encodes repeating glycyl-seryl sequences to provide flexible linkers between domains for maximizing independent activities of domains.

The expression constructs contain DNA that encodes specific chemical cleavage sites including, but not limited to, asparaginyl-glycyl or aspartyl-prolyl bonds (Bornstein and Balian, Methods Enzymol 47:132-145, 1977; Szoka, et al., DNA 5:11-20, 1986). The invention also provides for DNA that encodes specific protease cleavage sequences for Factor Xa or Enterokinase and the like (Jenny, et al., Protein Expr Purif 31:1-11, 2003; Wang, et al., Biol Chem Hoppe Seyler 376:681-684, 1995).

The expression constructs contain DNA that encodes an affinity “tag” sequence, for example, but not limited to, polyhistidine, V-5 epitope, or FLAG epitope to facilitate rapid, one-step purification of fusion proteins (Dobeli, et al., U.S. Pat. No. 5,047,513; Chen, et al., Eur J Biochem 214:845-852, 1993; Terpe, Appl Microbiol Biotechnol 60:523-533, 2003).

### Example 2

Purification of GBP-Fusion Proteins

Larger cultures were grown to produce sufficient fusion proteins for purification and characterization. To extract proteins under “native” conditions for subsequent purification, the bacteria were resuspended in 50 mM sodium phosphate buffer, pH 8.0, containing 0.5M sodium chloride and 10 mM imidazole to a final density approximately 20 times greater than that of the original cultures. Cells on ice were lysed by sonication at medium power and interval setting of 50% to give an intermittent pulse for 30 seconds. This was repeated for 6 cycles with one-minute rest on ice between cycles. Following each cycle, the optical density at 600 nm was recorded to assess cell lyses. The sonicated suspension was centrifuged 5,000×g for 10 min to remove cell debris and insoluble proteins from the soluble fraction. The resulting pellet was extracted in a “denaturing” solution of 20 mM sodium phosphate buffer, pH 7.8, containing 6M guanidine HCl (Gu-HCl) and 0.5M sodium chloride and the suspension was centrifuged to remove insoluble material.

In the case of the streptavidin fusion proteins, the cells were extracted only with 20 mM sodium phosphate buffer, pH7.8, containing 6M Gu-HCl and 0.5M sodium chloride.

The His6-tag recombinant proteins, were purified on ProBond nickel-resin columns (Invitrogen) as recommended by the manufacturer. Material in the two extracts, i.e., under native conditions for soluble proteins or denaturing conditions for insoluble proteins, was incubated with individual Probond Nickel resin columns, washed, and eluted as recommended by the manufacturer. Analysis by SDS-PAGE indicated that the final preparations were 90%-95% pure accompanied by proteolysis of a small amount of material, probably at the GBP domain. Initial extracts did not include protease inhibitors, but future preparations will include PMSF and a commercial “cocktail” of protease inhibitors. The optical density at 280 nm of the eluate fractions was recorded and the peak fractions from each column were pooled, aliquoted and stored at −20° C.

The inclusion of an Asn-Gly bond, susceptible to hydrolysis in 2M hydroxylamine and 4M urea at pH 9.5, allowed for physically dissociation of GBP from protein A as shown in FIG. 6. As a method to achieve limited digestion of proteins, urea is required to unfold proteins to make any Asn-Gly bonds fully accessible to hydroxylamine. However, because of the exposed location of our inserted Asn-Gly bond efficient hydrolysis was achieved without adding urea in just a few hours. Further, it was possible to hydrolyze the fusion protein while it was bound to gold powder. Thus, selectively hydrolyze fusion proteins is possible at the inserted Asn-Gly site even when fusion partners contain such bonds, especially if even less stringent conditions can be employed.

### Example 3

Thermo-stability of a GBP foundation layer on gold.

A study was conducted to determine the stability of GBP/gold complexes in comparison to bovine serum albumin (BSA)/gold complexes. The method introduces a confluent layer of GBP on spherical gold powder (Sigma-Aldrich, 1 to 3 microns in diameter). Control samples consisted of BSA/gold complexes or only gold powder. Samples in 1.0 mL of PKT buffer in Eppendorf centrifuge tubes were boiled in a water bath for 20 min with frequent mixing to maintain gold suspension, and 3 exchanges of buffer at 100° C. after gold collected at the bottom of the tubes. Gold samples were collected by centrifugation, washed 3× in PKT buffer, incubated with an amount of GBP-alkaline phosphatase (GBP-AP) sufficient to saturate the gold particles, washed 3× in PKT buffer, and assayed for alkaline phosphatase activity. Control samples of gold powder without prior incubation in GBP or BSA had GBP-AP binding capacity equal to 100%. GBP/gold and BSA/gold bound 3.7% and 29.3% GBP-AP, respectively, compared to control. This indicated that the GBP/gold complexes were extremely stable at high temperatures.

### Example 4

Chemical Resistance of a GBP Foundation Layer on Gold

Using the experimental approach described in EXAMPLE 3 above, two studies were conducted for 1.5 h or 72 h duration to assess the durability of GBP/gold complexes in strong chemical solutions. Gold samples were prepared with GBP or BSA layers or no layer, and incubated at room temperature with constant mixing in 1 mL of the following solvents or agents:

1.5 h incubations—0.1M NaOH with 1% Triton X-100, pH 12.8; 10% SDS; 6M Gu-HCl; 8M urea; 0.1M glycine-HCl, pH 2.2; 100% EtOH; 99% isopropanol; 2M NaCl.

72 h incubations—0.1M NaOH with 1% Triton X-100, pH 12.8; 10% SDS; 6M Gu-HCl; 8M urea; 100% methanol; 99% isopropanol; 10% acetic; 10% phosphoric acid; 10% sulfuric acid.

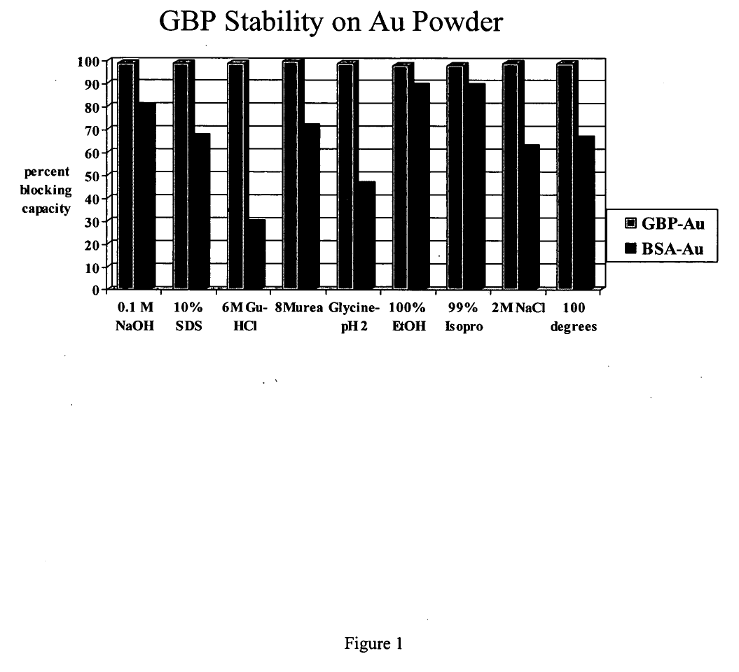

The results depicted in FIGS. 1 (1.5 h study) and 2 (72 h study) indicate the robust durability of GBP/gold complexes in strong solvents and chemical agents and extremes in pH compared to BSA/gold complexes. These observations support that GBP/gold complexes are extremely durable under extreme chemical conditions.

### Example 5

Construction and Characterization of Biosensors

Surface plasmon resonance (SPR)—an optical principle-biosensors were constructed on a fully integrated miniature SPR transducer, called Spreeta, from Texas Instruments (Melendez, et al., Sensors & Actuators B, 35, 36:212-216, 1996). Sensor chips were coated with recombinant His₆-protein A-GBP and His₆-streptavidin-GBP and the performance of each was compared to that of control sensors constructed with native protein A or recombinant streptavidin lacking the GBP domain. Solutions were delivered by a peristaltic pump at a flow rate of 0.2 mL/min at room temperature through a flow cell attached to each sensor. Clean sensing surfaces were rinsed initially for 10 min in 10 mM potassium phosphate buffer, pH 7.0 containing 10 mM potassium chloride and 1% Triton X-100 (PKT buffer) followed by solutions of PKT buffer containing test proteins. In the case of protein A-GBP or native protein A, the gold sensing surfaces were incubated for 10 min with 12 picomole of protein/mL For recombinant His₆-streptavidin-GBP or His₆-streptavidin 4.5 picomole of each/mL was used. Again, the presence of Gu-HCl precluded using higher amounts of protein.

### Example 6

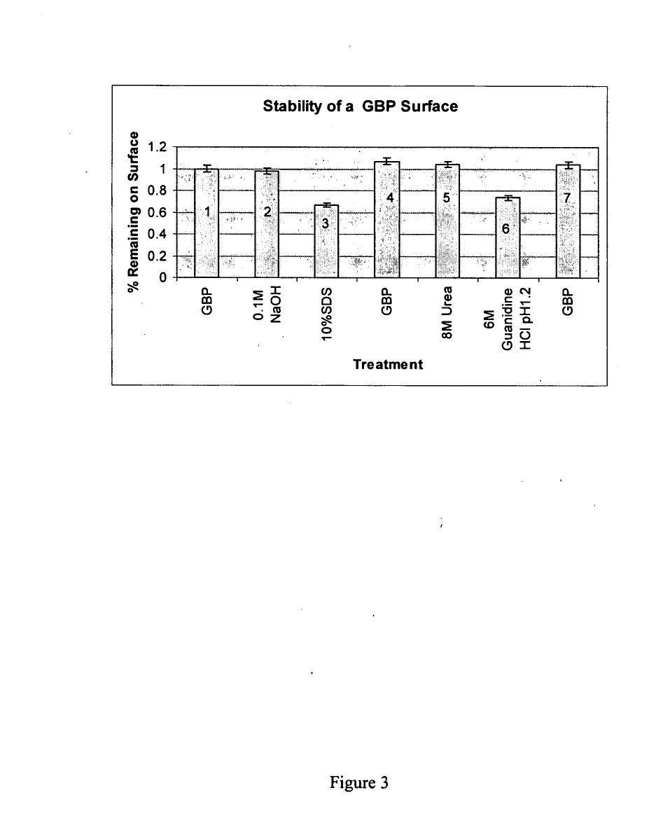

Stability the GBP Surface to Treatment with Known Protein Denaturants (FIG. 3).

A TI Spreeta sensor was used to monitor stability of GBP bound to its gold sensing surface. The sensor surface was first equilibrated under flow (120 ul/min) in reference buffer (PBSE). GBP was applied until surface saturation occurred. This was followed by the application of known protein destabilizing agents or additional GBP under identical flow conditions. The refractive index (RI) was monitored until stable values were obtained during each treatment as well as upon returning to reference buffer after each treatment. Error bars were computed from the standard deviations in the RI measurements.

Surface stability is the percent of GBP remaining on the surface and is computed by % Remaining=((RI(Treatment)−Baseline)/RI(GBP)−RI(Treatment))×100.

Wherein RI(Baseline) is the mean RI in reference buffer before treatment 1 (GBP), RI(treatment) is the mean RI in reference buffer following a given treatment and RI(GBP) is the mean RI in reference buffer following a GBP treatment. Percentages are computed vs. the first GBP (treatment 1) for treatments 1, 2, 3, and 4, and vs the second GBP (treatment 4) for treatments 5, 6, and 7. Application 0.1 M NaOH and 8M Urea did not affect the surface coverage whereas the application of 10% SDS and 6M Guanidine HCl resulted in surfaces retaining 67% and 73% of the bound GBP.

### Example 7

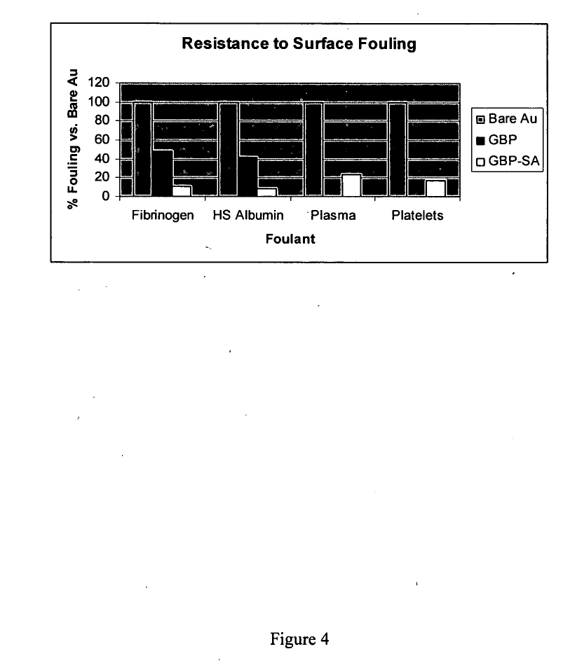

SPR Data Supporting the Non-Fouling Property of GBP-Streptavidin Coated Surface (FIG. 4).

Relative equilibrium response of sensor surfaces to the following physiological foulants: Fibrinogen(20 mg/ml), Human Serum Albumin (32 mg/ml), Plasma, and Platelets. Bare sensor surfaces and surfaces treated to saturation with either GBP or GBP-Streptavidin were equilibrated under flow with either fibrinogen, human serum albumin, human plasma or concentrated human platelets. 6 dual channel sensors were used. One channel in each was saturated with either GBP (2 sensors) or GBP-SA (4 sensors) while the other channel was exposed only to reference buffer (PBSE). Baselines for each channel were collected in reference buffer then both channels were exposed to foulant simultaneously until equilibrium was reached. Both channels were then rinsed in reference buffer until a stable response was obtained. In each sensor the change in refractive index in the bare gold channel was taken to be 100% fouling and the % fouling for the treated channels were computed as:

% Fouling=((RI(after foulant)−RI(before foulant))/(RI(bare surface, after foulant)−R(bare surface, before foulant)))×100. While GBP alone significantly reduces fouling of the gold surface 48 and 42% respectively for fibrinogen and human serum albumin fusion protein GBP-SA dramatically reduces fouling to 10% for fibrinogen, 8% for human serum albumin, 24% for plasma and 18% for platelets.

As depicted in FIG. 4 using SPR sensors, GBP alone blocked approximately 50% of surface fouling by concentrated levels of human serum fibrinogen or serum albumin. However, GBP-streptavidin blocked greater than 90% of each protein.

Relative equilibrium response of sensor surfaces to the following physiological foulants: Fibrinogen(20 mg/ml), Human Serum Albumin (32 mg/ml), Plasma, and Platelets. Bare sensor surfaces and surfaces treated to saturation with either GBP or GBP-Streptavidin were equilibrated under flow with either fibrinogen, human serum albumin, human plasma or platelet-enriched plasma. 6 dual channel sensors were used. One channel in each was saturated with either GBP (2 sensors) or GBP-SA (4 sensors) while the other channel was exposed only to reference buffer (PBSE). Baselines for each channel were collected in reference buffer then both channels were exposed to foulant simultaneously until equilibrium was reached. Both channels were then rinsed in reference buffer until a stable response was obtained. In each sensor the change in refractive index in the bare gold channel was taken to be 100% fouling and the % fouling for the treated channels were computed as: % Fouling=((RI(after foulant)−RI(before foulant))/(RI(bare surface, after foulant)−R(bare surface, before foulant)))×100. While GBP alone significantly reduces fouling of the gold surface 48 and 42% respectively for fibrinogen and human serum albumin fusion protein GBP-SA dramatically reduces fouling to 10% for fibrinogen, 8% for human serum albumin, 24% for plasma and 18% for platelets.

Therefore, GBP by itself does not fully block proteins from binding gold, but GBP-fusion proteins apparently are much better at blocking proteins. These proteins are the major source of surface fouling when biomaterials or biodetection devices are exposed to blood or plasma. Early fouling within seconds appears to occur initially by fibrinogen followed rapidly by serum albumin (Vroman and Adams, J Biomed Mater Res 3:43-67, 1969; Rudee and Price, J Biomed Mater Res 19:57-66,1998).

One possibility for the difference in resistance is that the small GBP molecule binds to gold in a random, string-like coil with little secondary or tertiary structure. When a gold surface is saturated with a monolayer of GBP there can be gaps exposing bare metal that can be fouled by proteins and other macromolecules in samples. When the GBP is fused to a relatively large, globular protein, however, that is positioned above the GBP layer, the fusion partner can block access to the bare gold.

Alternatively, there are few gaps exposing bare gold. Instead, non-specific binding of proteins and other macromolecules can occur at the epsilon amino groups of the abundant lysine residues on the GBP monolayer. The presence of streptavidin fusion partner can prevent the electrostatic interaction of macromolecules and GBP on gold.

### Example 8

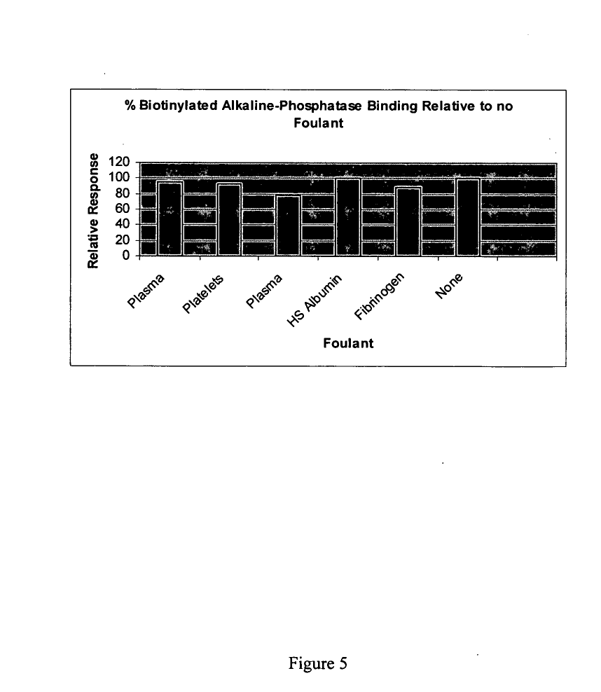

Relative Response of GBP-SA/GBP Channels After Exposure to Foulants (FIG. 5).

GBP-SA control channel. Experimental channels were created by saturating their surfaces with GBP-SA followed by a further saturation with GBP alone. Channels then were equilibrated with foulants under flow. After return to reference buffer the responses to 2 ug/ml biotinylated alkaline phosphatase (b-AP) were measured. Responses were computed as R(Experimental)=RI(After b-AP)−RI(Before b-AP). A control sensor was generated by saturating both channels with GBP-SA only then equilibrating with reference buffer and measuring the responses R(Control) to 2 ug/ml b-AP. This sensor provides maximal response to b-AP. Relative responses are computed as (R(Experimental)/R(Control))×100. All channels responded between 80-100% relative to the control hence the GBP-SA remains essentially fully active after fouling by common blood components.

The results demonstrate that 80% to 100% of the streptavidin activity was expressed after the sensors had been incubated in proteins or plasmas. Therefore, little fouling is apparent on sensors coated with GBP-streptavidin and the low amount of non-specific binding material detected does not obscure the bioactivity of streptavidin.

Although the invention has been described with reference to the above examples, it will be understood that modifications and variations are encompassed within the spirit and scope of the invention. Accordingly, the invention is limited only by the following claims.

What is claimed is:

1. A method of forming a biomolecular coating on a surface of a medical device comprising:
a) providing a medical device, wherein the device comprises one or more gold surfaces; and
b) applying a biomaterial to the device, wherein the biomaterial is adsorbed on or is formed on a surface thereof, and wherein the biomaterial comprises a fusion protein having at least one gold binding protein (GBP) domain and at least one proteinaceous biomolecule domain,
wherein applying the biomaterial immobilizes the biomolecule on the surface, thereby forming a biomolecular coating on the medical device.

2. The method of claim 1, wherein the biomolecule imparts biocompatibility characteristics to the surface of the device.

3. The method of claim 1, wherein the biomolecule promotes tissue healing and repair.

4. The method of claim 1, wherein the coating imparts resistance to fouling of the surface of the device.

5. The method of claim 1, wherein at least one biomolecule is selected from the group consisting of an anti-thrombotic protein, an anti-inflammatory protein, an antibody, an antigen, an immunoglobulin, an enzyme, a hormone, a neurotransmitter, a cytokine, a protein, a globular protein, a cell attachment protein, a peptide, a cell attachment peptide, a toxin, an antimicrobial protein, a cell receptor, an enzyme inhibitor, a polypeptide ligand, and a growth factor.

6. The method of claim 5, wherein the fusion protein comprises two or more GBP domains.

7. The method of claim 6, wherein the fusion protein comprises 7 GBP domains.

8. The method of claim 5, wherein each domain is separated by one or more peptide linkers of low complexity.

9. The method of claim 8, wherein the linkers comprise at least 5 amino acid residues.

10. The method of claim 9, wherein the linkers are repeating Gly-Ser residues.

11. The method of claim 10, wherein the linkers can be selectively hydrolyzed by enzymes or by chemical reaction.

12. The method of claim 5, wherein at least one biomolecule comprises an Arg-Gly-Asp (RGD) cellular adhesion consensus sequence.

13. The method of claim 5, wherein the biomolecule is bone sialoprotein (BSP) or osteopontin (OPN).

14. The method of claim 13, wherein the amino-carboxy terminus configuration for the domains is selected from the group consisting of GPB-OPN, OPN-GBP, GBP-BSP, and BSP-GBP.

15. The method of claim 5, wherein at least one biomolecules comprises a heparin binding consensus sequence as set forth in SEQ ID NO: 2.

16. The method of claim 5, wherein the biomolecule is a bone morphogenetic protein (BMP), a transforming growth factor (TGF), an osteonectin (ON), a fibronectin (FN), a fibroblast growth factor (FGF).

17. The method of claim 1, wherein the device is selected from the group consisting of a blood-contacting medical device, a tissue-contacting medical device, a bodily fluid-contacting medical device, an implantable medical device, an extracorporeal medical device, a dental device, a dental implant, a blood oxygenator, a blood pump, tubing for carrying blood, an endoprosthesis medical device, a vascular graft, a stent, a pacemaker lead, a heart valve, a temporary intravascular medical device, a catheter, nanoparticle, and a guide wire.

18. A tissue-interface device comprising at least one gold surface, which surface is routinely exposed to a tissue of a subject, and a biomaterial adsorbed on or formed on the surface to be exposed, wherein the biomaterial comprises a fusion protein having at least one gold binding protein (GBP) domain and at least one proteinaceous biomolecule domain, and wherein the adsorbed biomaterial immobilizes the biomolecule on the surface of the device.

19. The device of claim 18, wherein the biomolecule imparts biocompatibility characteristics to the surface of the device.

20. The device of claim 18, wherein the biomolecule promotes tissue healing and repair.

21. The device of claim 18, wherein the coating imparts resistance to fouling of the surface of the device.

22. The device of claim 18, wherein the fusion protein comprises two or more GBP domains.

23. The device of claim 22, wherein the fusion protein comprises 7 GBP domains.

24. The device of claim 18, wherein at least one biomolecule is selected from the group consisting of an anti-thrombotic protein, an anti-inflammatory protein, an antibody, an antigen, an immunoglobulin, an enzyme, a hormone, a neurotransmitter, a cytokine, a protein, a globular protein, a cell attachment protein, a peptide, a cell attachment peptide, a peptide toxin, an antimicrobial protein, a cell receptor, an enzyme inhibitor, a polypeptide ligand, and a growth factor.

25. The device of claim 24, wherein at least one biomolecule comprises an Arg-Gly-Asp (RGD) cellular adhesion consensus sequence.

26. The device of claim 25, wherein the biomolecule is bone sialoprotein (BSP) or osteopontin (OPN).

27. The device of claim 26, wherein the amino-carboxy terminus configuration for the domains is selected from the group consisting of GPB-OPN, OPN-GBP, GBP-BSP, and BSP-GBP.

28. The device of claim 18, wherein the device is selected from the group consisting of a blood-contacting medical device, a tissue-contacting medical device, a bodily fluid-contacting medical device, an implantable medical device, an extracorporeal medical device, a dental implant, a blood oxygenator, a blood pump, tubing for carrying blood, an endoprosthesis medical device, a vascular graft, a stent, a pacemaker lead, a heart valve, a temporary intravascular medical device, a catheter, a nanoparticle, and a guide wire.

29. A method of sterilizing a gold containing device comprising:
a) applying a biomaterial coating on the device, wherein the biomaterial is adsorbed on or is formed on a surface of the device, and wherein the biomaterial comprises a fusion protein having at least one gold binding protein (GBP) domain; and
b) sterilizing the coated device by a process comprising:
i) exposing the device to organic solutions selected from the group consisting of Gu-HCl, Triton X-100, methanol, ethanol, isopropanol, urea, acetic acid, and glycine-HCl,
ii) exposing the device to strong acids or bases,
iii) exposing the device to a temperature of about 100° C.,
iv) exposing the device to solutions of high ionic strength, or
v) a combination of processes (i)-(iv),
wherein the sterilizing does not significantly impact the adsorption of the GBP domain to the surface of the device.

30. The method of claim 29, wherein the GBP imparts biocompatibility characteristics to the surface of the device.

31. The method of claim 30, wherein the fusion protein comprises a thermophilic or extremophilic enzyme.

32. The method of claim 31, wherein the enzyme is selected from the group consisting of RNases, polymerases, restriction endonucleases, reductases, amino transferases, dismutases, synthases, amino peptidases, kinases, ligases, proteases, carboxypeptidases, phosphatases, binding proteins, amylases, pullulanases, amylopullulanases, glucoamylases, CGTases, glucanases, cellobiohydrolases, endoxylanases, mannanases, xylosidases, glucosidases, hydantoinases, esterases, aldolases, cytochrome P450, dehydrogenases, methylesterases, lyases, galactosidases, fructosidases, endoglucanases, phytases, keratinases, chitinases, and isomerases.

33. The method of claim 29, wherein the GBP imparts resistance to fouling of the surface of the device.

34. The method of claim 29, wherein the fusion protein comprises two or more GBP domains.

35. The method of claim 34, wherein the fusion protein comprises 7 GBP domains.

36. The method of claim 29, wherein the device is selected from the group consisting of a blood-contacting medical device, a tissue-contacting medical device, a bodily fluid-contacting medical device, an implantable medical device, an extracorporeal medical device, a dental implant, a blood oxygenator, a blood pump, tubing for carrying blood, an endoprosthesis medical device, a vascular graft, a stent, a pacemaker lead, a heart valve, a temporary intravascular medical device, a catheter, a bead, a biochip, a biosensor, and a guide wire.

37. A method of adsorbing a thermophilic or extremophilic enzyme to a gold containing surface comprising:
a) providing one or more gold surfaces; and
b) adsorbing a biomaterial on the one or more surfaces, wherein the biomaterial is adsorbed on or is formed on one or more surfaces, and wherein the biomaterial comprises a fusion protein having at least one gold binding protein (GBP) domain and at least one domain comprising a thermophilic or extremophilic enzyme,
wherein adsorbing the biomaterial immobilizes the thermophilic or extremophilic enzyme on the one or more surfaces.

38. The method of claim 37, wherein the surface is regularly exposed to temperature ranges from about 40° C. to about 100° C.

39. The method of claim 37, wherein the surface is selected from the group consisting of a bead, a microchip, an array, and a biosensor.

40. The method of claim 37, wherein the thermophilic enzyme is selected from the group consisting of RNases, polymerases, restriction endonucleases, reductases, amino transferases, dismutases, synthases, amino peptidases, kinases, ligases, proteases, carboxypeptidases, phosphatases, and binding proteins.

41. The method of claim 37, wherein the extremophilic enzyme is selected from the group consisting of amylases, pullulanases, amylopullulanases, glucoamylases, CGTase, glucanases, cellobiohydrolases, endoxylanases, mannanases, xylosidases, glucosidases, hydantoinases, esterases, aldolases, cytochrome P450, dehydrogenases, methylesterases, lyases, galactosidases, fructosidases, endoglucanases, phytases, keratinases, chitinases, and isomerases.

42. A gold containing device comprising a fusion protein adsorbed to one or more gold surfaces comprising the device, wherein the fusion protein comprises at least one gold binding protein (GBP) domain and at least one domain comprising a thermophilic or extremophilic enzyme, and wherein the GBP domain immobilizes the thermophilic or extremophilic enzyme on the surface of the device.

43. The device of claim 42, wherein the device is regularly exposed to temperature ranges from about 40° C. to about 100° C.

44. The device of claim 42, wherein the surface is selected from the group consisting of a bead, a microchip, an array, and a biosensor.

45. The device of claim 42, wherein the thermophilic enzyme is selected from the group consisting of RNases, polymerases, restriction endonucleases, reductases, amino transferases, dismutases, synthases, amino peptidases, kinases, ligases, proteases, carboxypeptidases, phosphatases, and binding proteins.

46. The device of claim 42, wherein the extremophilic enzyme is selected from the group consisting of amylases, pullulanases, amylopullulanases, glucoamylases, CGTase, glucanases, cellobiohydrolases, endoxylanases, mannanases, xylosidases, glucosidases, hydantoinases, esterases, aldolases, cytochrome P450, dehydrogenases, methylesterases, lyases, galactosidases, fructosidases, endoglucanases, phytases, keratinases, chitinases, and isomerases.

47. A method of producing a surface having a gold monolayer comprising:
a) applying a binding partner on a planar surface;
b) applying a fusion protein to the planar surface, wherein the fusion protein comprises a gold binding protein (GBP) domain and a protein domain, wherein the protein domain is a cognate binding partner to the applied binding partner of step (a); and
c) exposing the bound planar surface to one or more modalities comprising one or more gold surfaces, wherein the modalities are selected from the group consisting of gold comprising beads, colloidal gold, gold powder, and gold comprising nanoparticles,
wherein the interaction between the binding partner on the planar surface and cognate binding partner of the fusion protein drives the assembly of the modalities, thereby forming a gold comprising monolayer on the planar surface.

48. The method of claim 47, wherein the protein domain is selected from the group consisting of protein A, protein G, streptavidin, core streptavidin, neutravidin, avidin, avidin related protein 4/5, strep-tag, strep-tag II, an antibody, an antibody fragment, a single chain antibody, a receptor, and a peptide ligand.

49. The method of claim 47, wherein the binding partner on the planar surface is selected from the group consisting of protein A, protein G, streptavidin, core streptavidin, neutravidin, avidin, avidin related protein 4/5, strep-tag, strep-tag II, an antibody, an antibody fragment, a single chain antibody, biotin, receptor ligands, small molecules, nucleic acids, carbohydrates, lipids, inorganic compounds, organic compounds, vitamins, metals, and peptide ligands.

50. The method of claim 47, wherein the binding partner of step (a) is covalently bound to the planar surface.

51. The method of claim 47, wherein the binding partner is applied in a pattern.

52. The method of claim 47, wherein the planar surface is operatively coupled on a device.

53. The method of claim 52, wherein the device is a medical device, a microchip, a biochip, an array, or a biosensor.

54. A device produced by the method of claim 47.

55. The device of claim 54, wherein the fusion protein comprises two or more GBP domains.

56. The device of claim 55, wherein the fusion protein comprises 7 GBP domains.


SEQUENCE LISTING (61 sequences)

SEQ ID NO: 1 - protein, 14 aa, Artificial sequence
  Synthetic construct
MHGKTQATSGTIQS

SEQ ID NO: 2 - DNA, 16 bp, Artificial sequence
  Synthetic construct
tcgagggtccgtaata

SEQ ID NO: 3 - DNA, 16 bp, Artificial sequence
  Synthetic construct
agcttattacggaccc

SEQ ID NO: 4 - DNA, 24 bp, Artificial sequence
  Synthetic construct
ctggtagtggcaatggtcatatgc

SEQ ID NO: 5 - DNA, 25 bp, Artificial sequence
  Synthetic construct
tatgaccattgccactaccagagct

SEQ ID NO: 6 - protein, 27 aa, Artificial sequence
  Synthetic construct
TPVVPTVDTYDGRGDSVVYGLRSKSKK

SEQ ID NO: 7 - protein, 27 aa, Artificial sequence
  Synthetic construct
GYEIYESENGEPRGDNYRAYEDEYSYF

SEQ ID NO: 8 - protein, 27 aa, Artificial sequence
  Synthetic construct
TDYTAECKPQVTRGDVFTMPEDEYTVY

SEQ ID NO: 9 - protein, 27 aa, Artificial sequence
  Synthetic construct
VDYTITVYAVTGRGDSPASSKPISINY

SEQ ID NO: 10 - protein, 27 aa, Artificial sequence
  Synthetic construct
SHSLTTNIMEILRGDFSSANNRDNTYN

SEQ ID NO: 11 - protein, 27 aa, Artificial sequence
  Synthetic construct
YSKQFTSSTSYNRGDSTFESKSYKMAD

SEQ ID NO: 12 - protein, 27 aa, Artificial sequence
  Synthetic construct
PNPDQKDSDGDGRGDACKDDFDHDSVP

SEQ ID NO: 13 - protein, 27 aa, Artificial sequence
  Synthetic construct
PDTEYEVSLISRRGDMSSNPAKETFTT

SEQ ID NO: 14 - protein, 27 aa, Artificial sequence
  Synthetic construct
VGARGPAGPQGPRGDKGETGEQGDRGI

SEQ ID NO: 15 - protein, 27 aa, Artificial sequence
  Synthetic construct
RGAAGLPGPKGDRGDAGPKGADGSPGK

SEQ ID NO: 16 - protein, 27 aa, Artificial sequence
  Synthetic construct
AGPNGPPGPAGSRGDGGPPGMTGFPGA

SEQ ID NO: 17 - protein, 27 aa, Artificial sequence
  Synthetic construct
PGPAGKEGLRGPRGDQGPVGRTGEVGA

SEQ ID NO: 18 - protein, 27 aa, Artificial sequence
  Synthetic construct
VGPRGPSGPQGIRGDKGEPGEKGPRGL

SEQ ID NO: 19 - protein, 27 aa, Artificial sequence
  Synthetic construct
IILAALGGDVEKRGDREEAHVPFFSVM

SEQ ID NO: 20 - protein, 27 aa, Artificial sequence
  Synthetic construct
RGPPGGVGFPGSRGDTGPPGPPGYGPA

SEQ ID NO: 21 - protein, 27 aa, Artificial sequence
  Synthetic construct
KGDHGFPGSSGPRGDPGLKGDKGDVGL

SEQ ID NO: 22 - protein, 27 aa, Artificial sequence
  Synthetic construct
KGQIGPIGEKGSRGDPGTPGVPGKDGQ

SEQ ID NO: 23 - protein, 27 aa, Artificial sequence
  Synthetic construct
AYSPHPSLAKGARGDPGFPGAQGEPGS

SEQ ID NO: 24 - protein, 27 aa, Artificial sequence
  Synthetic construct
LPGEVLGAQPGPRGDAGVPGQPGLKGL

SEQ ID NO: 25 - protein, 27 aa, Artificial sequence
  Synthetic construct
PGIPGREGLPGDRGDPGDTGAPGPVGM

SEQ ID NO: 26 - protein, 27 aa, Artificial sequence
  Synthetic construct
PGPVGMKGLSGDRGDAGFTGEQGHPGS

SEQ ID NO: 27 - protein, 27 aa, Artificial sequence
  Synthetic construct
KGLAGEPGFKGSRGDPGPPGPPPVILP

SEQ ID NO: 28 - protein, 27 aa, Artificial sequence
  Synthetic construct
PGITGFPGFIGSRGDKGAPGRAGLYGE

SEQ ID NO: 29 - protein, 27 aa, Artificial sequence
  Synthetic construct
HGDPGFPGPPGERGDPGEANTLPGPVG

SEQ ID NO: 30 - protein, 27 aa, Artificial sequence
  Synthetic construct
QGRGGVSAVPGFRGDEGPIGHQGPIGQ

SEQ ID NO: 31 - protein, 27 aa, Artificial sequence
  Synthetic construct
RTQFTCECSIGFRGDGRTCYEVEKTRC

SEQ ID NO: 32 - DNA, 501 bp, Artificial sequence
  Synthetic construct
atgagaggatcgcatcaccatcaccatcacggatccggaggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatgctctgtccctcgagggttccggctccggttctggttctggtaccccggtcgtgccgacggtggacacctacgacggtcgcggcgactctgttgtttacggcctgcgcagcaagagcaaaaagttcgggtaa

SEQ ID NO: 33 - protein, 166 aa, Artificial sequence
  Synthetic construct
MRGSHHHHHHGSGGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKTQATSGTIQSMHALSLEGSGSGSGSGTPVVPTVDTYDGRGDSVVYGLRSKSKKFG

SEQ ID NO: 34 - DNA, 495 bp, Artificial sequence
  Synthetic construct
atgagaggatcgcatcaccatcaccatcacggatccggttctggttctggtaccccggtcgtgccgacggtggacacctacgacggtcgcggcgactctgttgtttacggcctgcgcagcaagagcaaaaagttcggttccggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggtccgtaa

SEQ ID NO: 35 - protein, 164 aa, Artificial sequence
  Synthetic construct
MRGSHHHHHHGSGSGSGTPVVPTVDTYDGRGDSVVYGLRSKSKKFGSGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGP

SEQ ID NO: 36 - DNA, 492 bp, Artificial sequence
  Synthetic construct
atgagaggatcgcatcaccatcaccatcacggatccggttctggttctggtggctacgagatctacgagagcgaaaacggtgagccgcgcggcgacaactatcgcgcatacgaggacgagtacagctatttcggttccggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggtccgtaa

SEQ ID NO: 37 - protein, 163 aa, Artificial sequence
  Synthetic construct
MRGSHHHHHHGSGSGSGGYEIYESENGEPRGDNYRAYEDEYSYFGSGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGP

SEQ ID NO: 38 - DNA, 597 bp, Artificial sequence
  Synthetic construct
atgagaggatcgcatcaccatcaccatcacggatccggttctggttctggtaccccggtcgtgccgacggtggacacctacgacggtcgcggcgactctgttgtttacggcctgcgcagcaagagcaaaaagttcggttccggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggttccggctccggttctggttctggtggctacgagatctacgagagcgaaaacggtgagccgcgcggcgacaactatcgcgcatacgaggacgagtacagctatttctaa

SEQ ID NO: 39 - protein, 198 aa, Artificial sequence
  Synthetic construct
MRGSHHHHHHGSGSGSGTPVVPTVDTYDGRGDSVVYGLRSKSKKFGSGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGSGSGSGSGGYEIYESENGEPRGDNYRAYEDEYSYF

SEQ ID NO: 40 - DNA, 600 bp, Artificial sequence
  Synthetic construct
atgagaggatcgcatcaccatcaccatcacggatccggttctggttctggtggctacgagatctacgagagcgaaaacggtgagccgcgcggcgacaactatcgcgcatacgaggacgagtacagctatttcggttccggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggttccggctccggttctggttctggtaccccggtcgtgccgacggtggacacctacgacggtcgcggcgactctgttgtttacggcctgcgcagcaagagcaaaaagttcgggtaa

SEQ ID NO: 41 - protein, 199 aa, Artificial sequence
  Synthetic construct
MRGSHHHHHHGSGSGSGGYEIYESENGEPRGDNYRAYEDEYSYFGSGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGSGSGSGSGTPVVPTVDTYDGRGDSVVYGLRSKSKKFG

SEQ ID NO: 42 - DNA, 495 bp, Artificial sequence
  Synthetic construct
atgagaggatcgcatcaccatcaccatcacggatccggaggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatgctctgtccctcgagggttccggctccggttctggttctggtggctacgagatctacgagagcgaaaacggtgagccgcgcggcgacaactatcgcgcatacgaggacgagtacagctatttctaa

SEQ ID NO: 43 - protein, 164 aa, Artificial sequence
  Synthetic construct
MRGSHHHHHHGSGGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKTQATSGTIQSMHALSLEGSGSGSGSGGYEIYESENGEPRGDNYRAYEDEYSYF

SEQ ID NO: 44 - DNA, 2205 bp, Artificial sequence
  Synthetic construct
atgagaggatcgcatcaccatcaccatcacggatccggttctggttctggtttcagcatgaaaaacctgcaccgccgcgtcaaaattgaagacagcgaagaaaatggcgtgttcaaataccgcccgcgttactacctgtacaagcacgcgtatttctacccgcacctgaagcgtttcccggttcagggctctagcgatagcagcgaggagaacggcgatgacagcagcgaggaggaagaggaagaagaggagaccagcaacgagggcgagaacaacgaagaaagcaatgaggacgaggacagcgaggccgagaacacgaccctgtctgcgaccaccctgggctacggtgaggacgccacgccaggcacgggctacacgggcctggcagcaattcaactgccgaagaaggccggcgatatcaccaacaaggcgaccaaggagaaggagagcgacgaggaggaagaagaggaggaggagggcaacgagaacgaggagagcgaggcagaagtggacgagaatgagcagggcatcaacggtaccagcacgaacagcacggaggcggagaatggcaatggttctagcggtggcgataatggtgaggagggcgaggaagaatctgtgacgggtgccaacgcagaaggcacgaccgagaccggcggtcagggcaagggcacctctaaaacgaccacgtctccgaatggcggtttcgagccaaccaccccgccgcaggtgtaccgtacgaccagcccgccgtttggcaagacgacgaccgtggagtacgaaggcgaatatgagtataccggtgtcaacgagtacgacaacggctacgagatctacgagagcgaaaacggtgagccgcgcggcgacaactatcgcgcatacgaggacgagtacagctatttcaagggccagggctatgatggctacgacggccagaactattaccaccaccagggttccggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggttccggatccggttctggttctggtataccggtcaagcaagcggatagcggcagcagcgaggaaaagcagctgtacaacaaatacccggacgcagtcgccacctggttaaacccagacccgagccagaagcagaatctgctggcgccgcaaaccctgccgagcaagagcaacgagagccacgaccacatggacgacatggacgacgaggacgatgacgaccacgttgacagccaagacagcatcgacagcaacgacagcgacgacgtggacgacacggacgattctcatcagagcgacgagtctcaccacagcgatgaaagcgacgagctggtgaccgactttccgacggatttaccagcgaccgaagtttttaccccggtcgtgccgacggtggacacctacgacggtcgcggcgactctgttgtttacggcctgcgcagcaagagcaaaaagttccgtcgtccagatattcagtatccggatgccacggatgaggatatcacctctcacatggagagcgaggagctgaacggcgcatacaaagcaatcccagtcgcccaagacctgaatgcgccaagcgactgggactctcgcggcaaagattcttacgagaccagccagctggatgaccaatctgccgagacccattctcacaagcagagccgcctgtacaagcgcaaagcgaacgatgaaagcaacgagcattctgacgtgatcgacagccaggagctgtctaaggtcagccgtgaattccatagccacgaattccacagccatgaggacatgctggtcgtggacccgaagagcaaggaggaggacaagcacctgaagtttcgcatcagccacgaactggacagcgctagctctgaggtcaatggttccggtgggagctcttaa

SEQ ID NO: 45 - protein, 734 aa, Artificial sequence
  Synthetic construct
MRGSHHHHHHGSGSGSGFSMKNLHRRVKIEDSEENGVFKYRPRYYLYKHAYFYPHLKRFPVQGSSDSSEENGDDSSEEEEEEEETSNEGENNEESNEDEDSEAENTTLSATTLGYGEDATPGTGYTGLAAIQLPKKAGDITNKATKEKESDEEEEEEEEGNENEESEAEVDENEQGINGTSTNSTEAENGNGSSGGDNGEEGEEESVTGANAEGTTETGGQGKGTSKTTTSPNGGFEPTTPPQVYRTTSPPFGKTTTVEYEGEYEYTGVNEYDNGYEIYESENGEPRGDNYRAYEDEYSYFKGQGYDGYDGQNYYHHQGSGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGSGSGSGSGIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVNGSGGSS

SEQ ID NO: 46 - DNA, 1263 bp, Artificial sequence
  Synthetic construct
atgagaggatcgcatcaccatcaccatcacggatccggttctggttctggtataccggtcaagcaagcggatagcggcagcagcgaggaaaagcagctgtacaacaaatacccggacgcagtcgccacctggttaaacccagacccgagccagaagcagaatctgctggcgccgcaaaccctgccgagcaagagcaacgagagccacgaccacatggacgacatggacgacgaggacgatgacgaccacgttgacagccaagacagcatcgacagcaacgacagcgacgacgtggacgacacggacgattctcatcagagcgacgagtctcaccacagcgatgaaagcgacgagctggtgaccgactttccgacggatttaccagcgaccgaagtttttaccccggtcgtgccgacggtggacacctacgacggtcgcggcgactctgttgtttacggcctgcgcagcaagagcaaaaagttccgtcgtccagatattcagtatccggatgccacggatgaggatatcacctctcacatggagagcgaggagctgaacggcgcatacaaagcaatcccagtcgcccaagacctgaatgcgccaagcgactgggactctcgcggcaaagattcttacgagaccagccagctggatgaccaatctgccgagacccattctcacaagcagagccgcctgtacaagcgcaaagcgaacgatgaaagcaacgagcattctgacgtgatcgacagccaggagctgtctaaggtcagccgtgaattccatagccacgaattccacagccatgaggacatgctggtcgtggacccgaagagcaaggaggaggacaagcacctgaagtttcgcatcagccacgaactggacagcgctagctctgaggtcaatggttccggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggtccgtaa

SEQ ID NO: 47 - protein, 420 aa, Artificial sequence
  Synthetic construct
MRGSHHHHHHGSGSGSGIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVNGSGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGP

SEQ ID NO: 48 - DNA, 1335 bp, Artificial sequence
  Synthetic construct
atgagaggatcgcatcaccatcaccatcacggatccggaggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggttccggatccggttctggttctggtttcagcatgaaaaacctgcaccgccgcgtcaaaattgaagacagcgaagaaaatggcgtgttcaaataccgcccgcgttactacctgtacaagcacgcgtatttctacccgcacctgaagcgtttcccggttcagggctctagcgatagcagcgaggagaacggcgatgacagcagcgaggaggaagaggaagaagaggagaccagcaacgagggcgagaacaacgaagaaagcaatgaggacgaggacagcgaggccgagaacacgaccctgtctgcgaccaccctgggctacggtgaggacgccacgccaggcacgggctacacgggcctggcagcaattcaactgccgaagaaggccggcgatatcaccaacaaggcgaccaaggagaaggagagcgacgaggaggaagaagaggaggaggagggcaacgagaacgaggagagcgaggcagaagtggacgagaatgagcagggcatcaacggtaccagcacgaacagcacggaggcggagaatggcaatggttctagcggtggcgataatggtgaggagggcgaggaagaatctgtgacgggtgccaacgcagaaggcacgaccgagaccggcggtcagggcaagggcacctctaaaacgaccacgtctccgaatggcggtttcgagccaaccaccccgccgcaggtgtaccgtacgaccagcccgccgtttggcaagacgacgaccgtggagtacgaaggcgaatatgagtataccggtgtcaacgagtacgacaacggctacgagatctacgagagcgaaaacggtgagccgcgcggcgacaactatcgcgcatacgaggacgagtacagctatttcaagggccagggctatgatggctacgacggccagaactattaccaccaccagggttccggtgggagctcttaa

SEQ ID NO: 49 - protein, 444 aa, Artificial sequence
  Synthetic construct
MRGSHHHHHHGSGGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGSGSGSGSGFSMKNLHRRVKIEDSEENGVFKYRPRYYLYKHAYFYPHLKRFPVQGSSDSSEENGDDSSEEEEEEEETSNEGENNEESNEDEDSEAENTTLSATTLGYGEDATPGTGYTGLAAIQLPKKAGDITNKATKEKESDEEEEEEEEGNENEESEAEVDENEQGINGTSTNSTEAENGNGSSGGDNGEEGEEESVTGANAEGTTETGGQGKGTSKTTTSPNGGFEPTTPPQVYRTTSPPFGKTTTVEYEGEYEYTGVNEYDNGYEIYESENGEPRGDNYRAYEDEYSYFKGQGYDGYDGQNYYHHQGSGGSS

SEQ ID NO: 50 - DNA, 1284 bp, Artificial sequence
  Synthetic construct
atgagaggatcgcatcaccatcaccatcacggatccggaggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggttccggatccggttctggttctggtataccggtcaagcaagcggatagcggcagcagcgaggaaaagcagctgtacaacaaatacccggacgcagtcgccacctggttaaacccagacccgagccagaagcagaatctgctggcgccgcaaaccctgccgagcaagagcaacgagagccacgaccacatggacgacatggacgacgaggacgatgacgaccacgttgacagccaagacagcatcgacagcaacgacagcgacgacgtggacgacacggacgattctcatcagagcgacgagtctcaccacagcgatgaaagcgacgagctggtgaccgactttccgacggatttaccagcgaccgaagtttttaccccggtcgtgccgacggtggacacctacgacggtcgcggcgactctgttgtttacggcctgcgcagcaagagcaaaaagttccgtcgtccagatattcagtatccggatgccacggatgaggatatcacctctcacatggagagcgaggagctgaacggcgcatacaaagcaatcccagtcgcccaagacctgaatgcgccaagcgactgggactctcgcggcaaagattcttacgagaccagccagctggatgaccaatctgccgagacccattctcacaagcagagccgcctgtacaagcgcaaagcgaacgatgaaagcaacgagcattctgacgtgatcgacagccaggagctgtctaaggtcagccgtgaattccatagccacgaattccacagccatgaggacatgctggtcgtggacccgaagagcaaggaggaggacaagcacctgaagtttcgcatcagccacgaactggacagcgctagctctgaggtcaatggttccggtgggagctcttaa

SEQ ID NO: 51 - protein, 427 aa, Artificial sequence
  Synthetic construct
MRGSHHHHHHGSGGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGSGSGSGSGIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVNGSGGSS

SEQ ID NO: 52 - DNA, 1314 bp, Artificial sequence
  Synthetic construct
atgagaggatcgcatcaccatcaccatcacggatccggttctggttctggtttcagcatgaaaaacctgcaccgccgcgtcaaaattgaagacagcgaagaaaatggcgtgttcaaataccgcccgcgttactacctgtacaagcacgcgtatttctacccgcacctgaagcgtttcccggttcagggctctagcgatagcagcgaggagaacggcgatgacagcagcgaggaggaagaggaagaagaggagaccagcaacgagggcgagaacaacgaagaaagcaatgaggacgaggacagcgaggccgagaacacgaccctgtctgcgaccaccctgggctacggtgaggacgccacgccaggcacgggctacacgggcctggcagcaattcaactgccgaagaaggccggcgatatcaccaacaaggcgaccaaggagaaggagagcgacgaggaggaagaagaggaggaggagggcaacgagaacgaggagagcgaggcagaagtggacgagaatgagcagggcatcaacggtaccagcacgaacagcacggaggcggagaatggcaatggttctagcggtggcgataatggtgaggagggcgaggaagaatctgtgacgggtgccaacgcagaaggcacgaccgagaccggcggtcagggcaagggcacctctaaaacgaccacgtctccgaatggcggtttcgagccaaccaccccgccgcaggtgtaccgtacgaccagcccgccgtttggcaagacgacgaccgtggagtacgaaggcgaatatgagtataccggtgtcaacgagtacgacaacggctacgagatctacgagagcgaaaacggtgagccgcgcggcgacaactatcgcgcatacgaggacgagtacagctatttcaagggccagggctatgatggctacgacggccagaactattaccaccaccagggttccggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggtccgtaa

SEQ ID NO: 53 - protein, 437 aa, Artificial sequence
  Synthetic construct
MRGSHHHHHHGSGSGSGFSMKNLHRRVKIEDSEENGVFKYRPRYYLYKHAYFYPHLKRFPVQGSSDSSEENGDDSSEEEEEEEETSNEGENNEESNEDEDSEAENTTLSATTLGYGEDATPGTGYTGLAAIQLPKKAGDITNKATKEKESDEEEEEEEEGNENEESEAEVDENEQGINGTSTNSTEAENGNGSSGGDNGEEGEEESVTGANAEGTTETGGQGKGTSKTTTSPNGGFEPTTPPQVYRTTSPPFGKTTTVEYEGEYEYTGVNEYDNGYEIYESENGEPRGDNYRAYEDEYSYFKGQGYDGYDGQNYYHHQGSGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGP

SEQ ID NO: 54 - DNA, 2205 bp, Artificial sequence
  Synthetic construct
atgagaggatcgcatcaccatcaccatcacggatccggttctggttctggtataccggtcaagcaagcggatagcggcagcagcgaggaaaagcagctgtacaacaaatacccggacgcagtcgccacctggttaaacccagacccgagccagaagcagaatctgctggcgccgcaaaccctgccgagcaagagcaacgagagccacgaccacatggacgacatggacgacgaggacgatgacgaccacgttgacagccaagacagcatcgacagcaacgacagcgacgacgtggacgacacggacgattctcatcagagcgacgagtctcaccacagcgatgaaagcgacgagctggtgaccgactttccgacggatttaccagcgaccgaagtttttaccccggtcgtgccgacggtggacacctacgacggtcgcggcgactctgttgtttacggcctgcgcagcaagagcaaaaagttccgtcgtccagatattcagtatccggatgccacggatgaggatatcacctctcacatggagagcgaggagctgaacggcgcatacaaagcaatcccagtcgcccaagacctgaatgcgccaagcgactgggactctcgcggcaaagattcttacgagaccagccagctggatgaccaatctgccgagacccattctcacaagcagagccgcctgtacaagcgcaaagcgaacgatgaaagcaacgagcattctgacgtgatcgacagccaggagctgtctaaggtcagccgtgaattccatagccacgaattccacagccatgaggacatgctggtcgtggacccgaagagcaaggaggaggacaagcacctgaagtttcgcatcagccacgaactggacagcgctagctctgaggtcaatggttccggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggttccggatccggttctggttctggtttcagcatgaaaaacctgcaccgccgcgtcaaaattgaagacagcgaagaaaatggcgtgttcaaataccgcccgcgttactacctgtacaagcacgcgtatttctacccgcacctgaagcgtttcccggttcagggctctagcgatagcagcgaggagaacggcgatgacagcagcgaggaggaagaggaagaagaggagaccagcaacgagggcgagaacaacgaagaaagcaatgaggacgaggacagcgaggccgagaacacgaccctgtctgcgaccaccctgggctacggtgaggacgccacgccaggcacgggctacacgggcctggcagcaattcaactgccgaagaaggccggcgatatcaccaacaaggcgaccaaggagaaggagagcgacgaggaggaagaagaggaggaggagggcaacgagaacgaggagagcgaggcagaagtggacgagaatgagcagggcatcaacggtaccagcacgaacagcacggaggcggagaatggcaatggttctagcggtggcgataatggtgaggagggcgaggaagaatctgtgacgggtgccaacgcagaaggcacgaccgagaccggcggtcagggcaagggcacctctaaaacgaccacgtctccgaatggcggtttcgagccaaccaccccgccgcaggtgtaccgtacgaccagcccgccgtttggcaagacgacgaccgtggagtacgaaggcgaatatgagtataccggtgtcaacgagtacgacaacggctacgagatctacgagagcgaaaacggtgagccgcgcggcgacaactatcgcgcatacgaggacgagtacagctatttcaagggccagggctatgatggctacgacggccagaactattaccaccaccagggttccggtgggagctcttaa

SEQ ID NO: 55 - protein, 734 aa, Artificial sequence
  Synthetic construct
MRGSHHHHHHGSGSGSGIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVNGSGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGSGSGSGSGFSMKNLHRRVKIEDSEENGVFKYRPRYYLYKHAYFYPHLKRFPVQGSSDSSEENGDDSSEEEEEEEETSNEGENNEESNEDEDSEAENTTLSATTLGYGEDATPGTGYTGLAAIQLPKKAGDITNKATKEKESDEEEEEEEEGNENEESEAEVDENEQGINGTSTNSTEAENGNGSSGGDNGEEGEEESVTGANAEGTTETGGQGKGTSKTTTSPNGGFEPTTPPQVYRTTSPPFGKTTTVEYEGEYEYTGVNEYDNGYEIYESENGEPRGDNYRAYEDEYSYFKGQGYDGYDGQNYYHHQGSGGSS

SEQ ID NO: 56 - DNA, 1323 bp, Artificial sequence
  Synthetic construct
atgagaggatcgcatcaccatcaccatcacggatccggttctggttctggtttccaccgccgcattaaagccggttccggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggttccggatccggttctggttctggtataccggtcaagcaagcggatagcggcagcagcgaggaaaagcagctgtacaacaaatacccggacgcagtcgccacctggttaaacccagacccgagccagaagcagaatctgctggcgccgcaaaccctgccgagcaagagcaacgagagccacgaccacatggacgacatggacgacgaggacgatgacgaccacgttgacagccaagacagcatcgacagcaacgacagcgacgacgtggacgacacggacgattctcatcagagcgacgagtctcaccacagcgatgaaagcgacgagctggtgaccgactttccgacggatttaccagcgaccgaagtttttaccccggtcgtgccgacggtggacacctacgacggtcgcggcgactctgttgtttacggcctgcgcagcaagagcaaaaagttccgtcgtccagatattcagtatccggatgccacggatgaggatatcacctctcacatggagagcgaggagctgaacggcgcatacaaagcaatcccagtcgcccaagacctgaatgcgccaagcgactgggactctcgcggcaaagattcttacgagaccagccagctggatgaccaatctgccgagacccattctcacaagcagagccgcctgtacaagcgcaaagcgaacgatgaaagcaacgagcattctgacgtgatcgacagccaggagctgtctaaggtcagccgtgaattccatagccacgaattccacagccatgaggacatgctggtcgtggacccgaagagcaaggaggaggacaagcacctgaagtttcgcatcagccacgaactggacagcgctagctctgaggtcaatggttccggtgggagctcttaa

SEQ ID NO: 57 - protein, 440 aa, Artificial sequence
  Synthetic construct
MRGSHHHHHHGSGSGSGFHRRIKAGSGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGSGSGSGSGIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVNGSGGSS

SEQ ID NO: 58 - DNA, 1374 bp, Artificial sequence
  Synthetic construct
atgagaggatcgcatcaccatcaccatcacggatccggttctggttctggtttccaccgccgcattaaagccggttccggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggttccggatccggttctggttctggtttcagcatgaaaaacctgcaccgccgcgtcaaaattgaagacagcgaagaaaatggcgtgttcaaataccgcccgcgttactacctgtacaagcacgcgtatttctacccgcacctgaagcgtttcccggttcagggctctagcgatagcagcgaggagaacggcgatgacagcagcgaggaggaagaggaagaagaggagaccagcaacgagggcgagaacaacgaagaaagcaatgaggacgaggacagcgaggccgagaacacgaccctgtctgcgaccaccctgggctacggtgaggacgccacgccaggcacgggctacacgggcctggcagcaattcaactgccgaagaaggccggcgatatcaccaacaaggcgaccaaggagaaggagagcgacgaggaggaagaagaggaggaggagggcaacgagaacgaggagagcgaggcagaagtggacgagaatgagcagggcatcaacggtaccagcacgaacagcacggaggcggagaatggcaatggttctagcggtggcgataatggtgaggagggcgaggaagaatctgtgacgggtgccaacgcagaaggcacgaccgagaccggcggtcagggcaagggcacctctaaaacgaccacgtctccgaatggcggtttcgagccaaccaccccgccgcaggtgtaccgtacgaccagcccgccgtttggcaagacgacgaccgtggagtacgaaggcgaatatgagtataccggtgtcaacgagtacgacaacggctacgagatctacgagagcgaaaacggtgagccgcgcggcgacaactatcgcgcatacgaggacgagtacagctatttcaagggccagggctatgatggctacgacggccagaactattaccaccaccagggttccggtgggagctcttaa

SEQ ID NO: 59 - protein, 457 aa, Artificial sequence
  Synthetic construct
MRGSHHHHHHGSGSGSGFHRRIKAGSGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGSGSGSGSGFSMKNLHRRVKIEDSEENGVFKYRPRYYLYKHAYFYPHLKRFPVQGSSDSSEENGDDSSEEEEEEEETSNEGENNEESNEDEDSEAENTTLSATTLGYGEDATPGTGYTGLAAIQLPKKAGDITNKATKEKESDEEEEEEEEGNENEESEAEVDENEQGINGTSTNSTEAENGNGSSGGDNGEEGEEESVTGANAEGTTETGGQGKGTSKTTTSPNGGFEPTTPPQVYRTTSPPFGKTTTVEYEGEYEYTGVNEYDNGYEIYESENGEPRGDNYRAYEDEYSYFKGQGYDGYDGQNYYHHQGSGGSS

SEQ ID NO: 60 - DNA, 432 bp, Artificial sequence
  Synthetic construct
atgagaggatcgcatcaccatcaccatcacggatccggttctggttctggtttccaccgccgcattaaagccggttccggtgggagctctggtagtggcaatggtcatatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagcatgcatgctctgtccctcgagggtccgtaa

SEQ ID NO: 61 - protein, 143 aa, Artificial sequence
  Synthetic construct
MRGSHHHHHHGSGSGSGFHRRIKAGSGGSSGSGNGHMHGKTQATSGTIQSMHGKTQATSGTIQSMHGKTQATSGTIQSMHGKTQATSGTIQSMHGKTQATSGTIQSMHGKTQATSGTIQSMHGKIQATSGTIQSMHALSLEGP